(19) United States
(12) Patent Application Publication
(10) Pub. No.: US 2019/0069931 A1
(43) Pub. Date: Mar. 7, 2019

(54) MODULAR PEDICLE SCREWS, MODULAR PEDICLE SCREW ASSEMBLIES, AND ASSOCIATED METHODS

(71) Applicant: LOMA LINDA UNIVERSITY, Loma Linda, CA (US)

(72) Inventor: Serkan INCEOGLU, Moreno Valley, CA (US)

(73) Assignee: LOMA LINDA UNIVERSITY, Loma Linda, CA (US)

(21) Appl. No.: 16/178,196

(22) Filed: Nov. 1, 2018

Related U.S. Application Data
(63) Continuation-in-part of application No. PCT/US2018/025300, filed on Mar. 29, 2018.
(60) Provisional application No. 62/479,285, filed on Mar. 30, 2017.

(51) Int. Cl. A61B 17/70; A61B 17/86

(52) U.S. Cl. CPC A61B 17/7032; A61B 17/7037; A61B 17/8685; A61B 17/8625; A61B 17/8605; A61B 17/7035

(57) ABSTRACT

A pedicle screw and pedicle screw assembly for use in supporting a spinal rod, the pedicle screw including a threaded shank, a screw head, and a screw neck connecting the threaded shank and the screw head. The screw head can include an arcuate upper surface to engage a lower surface of a modular head. A pair of arcuate channels positioned on either side of the screw head can receive a bottom flange portion of the modular head therein so as to secure the modular head to the pedicle screw. An upper surface of the modular head can define a rod recess for receiving the spinal rod. The relative configuration of the screw head and the modular head can be arranged so that once engaged, the modular head is freely slidable along the arcuate upper surface of the screw head, but the screw head and the modular head remain coupled.

### CROSS-REFERENCE TO RELATED APPLICATIONS

The present application is a Continuation in Part of and claims priority to International Application No. PCT/US2018/025300, filed Mar. 29, 2018, titled “MODULAR PEDICLE SCREW ASSEMBLIES AND ASSOCIATED METHODS,” which claims priority to, and the benefit of, U.S. Provisional Patent Application No. 62/479,285, filed Mar. 30, 2017, titled “MODULAR PEDICLE SCREW ASSEMBLIES AND ASSOCIATED METHODS,” each of which is incorporated herein by reference in its entirety.

### TECHNICAL FIELD

The present disclosure relates to surgical spine treatment and methods and, more particularly, modular pedicle screws, modular pedicle screw assemblies, and associated methods used in the field of surgical spine treatment.

### BACKGROUND

Surgical techniques for the treatment of spinal injuries or deformities often utilize a process called “spinal fusion,” which joins together two or more vertebrae of the spine. One method of spinal fusion utilizes a fixation system that is anchored to the spine with orthopedic screws that are implanted into pedicles of two or more adjacent vertebrae. The single screws may be connected together with rigid or semi-rigid rods, which rest housed within a transversal channel provided in the screw head. The screws are called pedicle screws, which can be, but are not always, inserted percutaneously (through the skin) into the pedicle of the vertebra. These screws may then be threaded into the bone. After the screws are threaded into place in the pedicle, metal rods are inserted to connect the screws and provide stability to the spine during the fusion process. Typically, the surgeon will use a bone graft to facilitate fusion.

Pedicle screws are generally used in the lumbar (low back) spine, but they can also be used in the thoracic (mid-back), cervical spine (neck) and sacral vertebrae. Typical procedures that use pedicle screws include: transforaminal lumbar interbody fusion (TLIF), posterior lumbar interbody fusion (PLIF), lateral lumbar interbody fusion (LLIF), and anterior lumbar interbody fusion (ALIF).

Due to the irregularity of bone anatomy and various curvatures of the spine, and the difficulties of accurate placement particularly in minimally invasive surgery, it is unlikely that the heads of the screws will be properly aligned for rod insertion after the screws have been implanted into the spine pedicles. Proper screw alignment and orientation allows for placement of the rods so that the stress of the rod is evenly distributed among the screws. If one screw assembly is out of place (misaligned), the rod will unevenly stress the various screw assemblies, which often leads to breakage of the overstressed screw or can also result in the screw loosening (i.e., loss of screw-bone engagement). For example, in some instances the surgeon may have to back the screw out of the pedicle to align the channel or to raise the screw to match the rod level. Both of these maneuvers weaken the bone-screw engagement. In addition, bending the rod too much to match the screw heads may also jeopardize the rod's long-term strength. Instead, in order to facilitate the insertion of the rod and to provide proper alignment of the rod (to alleviate stress on the screws), the screws may often be provided with a head that is freely rotatable with respect to the shank of each screw. This way, after insertion of the screw into the pedicle and before placement of the rod, the surgeon may rotate the various screw heads to achieve the desired alignment to receive the rod.

Currently this screw and rod alignment may be achieved with a poly-axial screw, in which the screw head and shank may be connected via a “ball and socket” mechanism that allows the upper part of the screw to swivel. The upper part of the socket-like cavity may have a locking insert used to clamp the spherical head once the appropriate orientation of the shank has been set. The transversal channel (referred to as a U-shaped channel) for housing the connecting rod may be arranged above the socket-like cavity, and a set-screw may be inserted above the rod in order to clamp the rod into position. Poly-axial screws are an improvement over the uniaxial screws, but the mobility of the head provided by a poly-axial screw requires a reduction in the shank diameter at the shank-head junction. Considering that pedicle screws sustain the largest stress at the shank-head junction, the reduction of the diameter at this region weakens the overall strength of the screw, such that the poly-axial screw introduces further mechanical drawbacks.

### SUMMARY

In view of the foregoing, Applicant has recognized that the diameter reduction in the shank can be prevented by increasing the size of the spherical head; however, there is a risk that this construction may lead to a bulky screw design which could impede the usability of the screw during surgery. A smaller shaft also may result in breakage. Accordingly, Applicant also has recognized that a pedicle screw assembly that maintains a larger, consistent diameter along the screw shaft, but also allows rotation at the head and provides the surgeon with the ability to fine tune the alignment of the assembly is desirable. Embodiments according to the present disclosure meet this need, as well as provide additional advantages such as deformity correction and ease of maintenance.

One aspect of the present technology provides a pedicle screw for use in supporting a modular head and a spinal rod connected to the modular head. The pedicle screw can include a threaded shank, a screw head positioned at an upper end portion of the threaded shank, and a screw neck connecting the threaded shank and the screw head. The screw head can include an arcuate upper surface forming a convex slide positioned to engage a pair of rails positioned on a lower surface of the modular head. The screw head can further include a pair of arcuate channels, each of the pair of arcuate channels positioned below the arcuate upper surface on an opposite side of the screw head, each of the pair of arcuate channels positioned to receive a bottom flange portion of the pair of rails of the modular head therein so as to secure the modular head on the pedicle screw. The screw neck can have a circular cross-section and can be positioned to provide a tapered transition between the screw head and the threaded shank.

Another aspect of the present technology provides a pedicle screw assembly for use in supporting a spinal rod. The pedicle screw assembly includes a pedicle screw having a threaded shank, a screw head positioned at an upper end portion of the threaded shank, and a screw neck connecting the threaded shank and the screw head. The screw head can include an arcuate upper surface forming a convex slide positioned to engage a pair of rails positioned on a lower surface of a modular head, and a pair of arcuate channels, each of the pair of arcuate channels positioned below the arcuate upper surface on an opposite side of the screw head, each of the pair of arcuate channels positioned to receive a bottom flange portion of the pair of rails of the modular head therein so as to secure the modular head on the pedicle screw. The screw neck connecting the threaded shank and the screw head can have a circular cross-section and positioned to provide a tapered transition between the screw head and the threaded shank. The pedicle screw assembly can also include a modular head having a rod recess positioned on an upper surface of the modular head so as to receive the spinal rod, and the lower surface of the modular head being positioned to define the bottom flange portion for engagement with the screw head. In addition, the relative configuration of the screw head and the modular head can be arranged so that once engaged, the modular head is freely slidable along the arcuate upper surface of the screw head, but the screw head and the modular head remain coupled to one another.

Yet another aspect of the present technology provides a method of placing a spinal rod. The method can include the steps of threading a pedicle screw shaft into a vertebrae, the pedicle screw shaft having a screw head positioned at an upper end portion thereof, the screw head having an arcuate upper surface forming a convex slide positioned to engage a pair of rails positioned on a lower surface of a modular head. The method can further include attaching the modular head to the head of the pedicle screw so that the modular head is freely slidable along the arcuate upper surface of the screw head in a direction determined by the curvature of the pair of rails engaged with the convex slide. The method further includes the steps of orienting the modular head so that a rod receiver recess positioned on an upper surface of the modular head is positioned to support the spinal rod, and inserting the spinal rod into the rod receiver recess.

In some embodiments, the step of threading a pedicle screw shaft into a vertebrae can include threading a plurality of pedicle screw shafts into a vertebrae, and the step of attaching the modular head to the head of the pedicle screw can include attaching a modular head to a pedicle screw shaft associated with each of the plurality of pedicle screw shafts. In addition, the step of orienting the head so that a rod receiver recess is positioned to support a spinal rod can include orienting each head so that the rod receiver recess is aligned with the rod receiver recess of an adjacent head.

In some embodiments, the method can also include the step of placing the spinal rod into the rod receiver recesses of a plurality of modular heads. In addition, the method can include installing a set screw in the head to fix the spinal rod relative to the head.

An additional embodiment of the present disclosure is directed to a modular pedicle screw assembly kit for stabilizing vertebrae of a spine. The kit can include a container; a pedicle screw positioned in the container; a modular head positioned the container; and a spinal rod positioned in the container. The pedicle screw can have a threaded shank, a screw head positioned at an upper end of the threaded shank, and a screw neck connecting the threaded shank and the screw head. The screw head can include an arcuate upper surface forming a convex slide positioned to engage a pair of rails positioned on a lower surface of a modular head, and a pair of arcuate channels. Each of the pair of arcuate channels can be positioned below the arcuate upper surface on an opposite side of the screw head, and each of the pair of arcuate channels can be positioned to receive a bottom flange portion of the pair of rails of the modular head therein so as to secure the modular head on the pedicle screw. The screw neck can have a circular cross-section and can be positioned to provide a tapered transition between the screw head and the threaded shank. The modular head can have a rod recess positioned on an upper surface of the modular head so as to receive the spinal rod, and the lower surface of the modular head can be positioned so as to define the bottom flange portion for engagement with the screw head.

### BRIEF DESCRIPTION OF THE FIGURES

The present technology will be better understood on reading the following detailed description of non-limiting embodiments thereof, and on examining the accompanying drawings, in which:

FIG. 1 is a perspective view of a plurality of modular pedicle screw assemblies with rods and locking mechanisms according to embodiments of the present technology.

FIG. 2 is an exploded perspective view of a modular pedicle screw assembly accordingly to an embodiment of the present technology.

FIG. 3A is a front or rear elevational view of the base portion of a modular pedicle screw assembly according to an embodiment of the present technology.

FIG. 3B is a side perspective view of the base portion of a modular pedicle screw assembly according to an embodiment of the present technology.

FIG. 3C is a left side elevational view of the base portion of a modular pedicle screw assembly according to an embodiment of the present technology.

FIG. 4A is a front or rear elevational view of the tulip head according to an embodiment of the present technology.

FIG. 4B is a left side elevational view of the tulip head according to an embodiment of the present technology.

FIG. 4C is a perspective view from the top looking down of the tulip head according to an embodiment of the present technology.

FIG. 4D is a perspective view from the bottom looking up of the tulip head according to an embodiment of the present technology.

FIG. 5A is a perspective view of a modular pedicle screw assembly according to an embodiment of the present technology.

FIG. 5B is a left side elevational view of a modular pedicle screw assembly according to an embodiment of the present technology.

FIG. 6A is a perspective view of a parallel orientated U-shaped channel tulip head having a U-shaped channel orientated in the tulip head so that the channel runs parallel with the rails, according to an embodiment of the present technology.

FIG. 6B is a perspective view of a perpendicular orientated U-shaped channel tulip head which has a U-shaped channel orientated in the tulip head so that the channel runs perpendicular with the rails, according to an embodiment of the present technology.

FIG. 7A is a left side elevational view of the dual U channel tulip head, showing the channel that runs parallel with the rails according to an embodiment of the present technology.

FIG. 7B shows a left side elevational view of the dual U-channel tulip head, showing the channel that runs perpendicular to the rails according to an embodiment of the present technology.

FIG. 7C shows a perspective view of the dual U channel tulip head according to an embodiment of the present technology.

FIG. 7D shows a top view of the dual U channel tulip head according to an embodiment of the present technology.

FIGS. 8A-8D provide four depictions of a dual U-channel tulip head rotated to accommodate different orientations to receive the rod according to an embodiment of the present technology.

FIG. 9A is a two part, rotatable tulip head having an upper section comprising the U-shaped channel and a lower section comprising the rails, and shows that the two part, rotatable tulip head may be positioned along the base 201, according to an embodiment of the present technology.

FIG. 9B shows the two-part, rotatable tulip head rotated about the lower section 375, according to an embodiment of the present technology.

FIGS. 9C-9E show perspective views illustrating that the lower section may be designed to receive the upper section (i.e., the male end is identified by reference numeral and the female end is identified by reference numeral), according to an embodiment of the present disclosure.

FIG. 10 is a flow chart depicting steps in a method of achieving alignment/orientation of the U-shaped channels in the modular pedicle screw assemblies used in spinal stabilization surgery according to an embodiment of the present technology.

FIG. 11 is a flow chart depicting steps in a method of achieving alignment/orientation of the U-shaped channels in the modular pedicle screw assemblies used in spinal stabilization surgery according to an embodiment of the present technology.

FIGS. 12A-12C show an embodiment of the present disclosure in which a series of pedicle screws are not aligned perfectly on a single plane but are instead tilted and offset with respect to each other to accommodate curvature of the spine.

FIGS. 13A-13B show an embodiment of the present disclosure in which the pedicle screws are placed in an imperfect or irregular alignment or orientation, and not in a single plane.

FIG. 14 is a perspective view of a plurality of modular pedicle screw assemblies with rods and locking mechanisms according to an embodiment of the present technology.

FIG. 15 is an exploded perspective view of a modular pedicle screw assembly according to an embodiment of the present technology.

FIG. 16 is a perspective view of a modular pedicle screw assembly according to an embodiment of the present technology.

FIG. 17 is a left side elevational view of a modular pedicle screw assembly according to an embodiment of the present technology.

FIG. 18 is a right side elevational view of a modular pedicle screw assembly according to an embodiment of the present technology.

FIG. 19 is a front elevational view of a modular pedicle screw assembly according to an embodiment of the present technology.

FIG. 20 is a rear elevational view of a modular pedicle screw assembly according to an embodiment of the present technology.

FIG. 21 is an off-center elevational view of a modular pedicle screw assembly according to an alternate embodiment of the present technology.

FIG. 22 is a cross-sectional view of a base of a modular screw assembly, including a ring, according to an embodiment of the present technology.

FIG. 23 is a perspective view of a ring for use in the base shown in FIG. 22.

FIG. 24 is a perspective view of a screw head according to an embodiment of the present technology.

FIG. 25 A is a cross-sectional view of a modular pedicle screw assembly according to the embodiment shown in FIG. 21.

FIG. 26 is an exploded perspective view of a modular pedicle screw assembly according to an embodiment of the present technology.

FIG. 27 is a perspective view of a modular pedicle screw assembly according to an embodiment of the present technology.

FIG. 28 is a left side elevational view of a modular pedicle screw assembly according to an embodiment of the present technology.

FIG. 29 is a right side elevational view of a modular pedicle screw assembly according to an embodiment of the present technology.

FIG. 30 is a front elevational view of a modular pedicle screw assembly according to an embodiment of the present technology.

FIG. 31 is a rear elevational view of a modular pedicle screw assembly according to an alternate embodiment of the present technology.

FIG. 32 is an off-center elevational view of a pedicle screw according to an embodiment of the present technology.

FIG. 33 is a perspective view of a simplified modular pedicle screw assembly kit according to an embodiment of the present technology.

### DETAILED DESCRIPTION

Embodiments of the present disclosure will now be described more fully hereinafter with reference to the accompanying drawings, in which preferred embodiments of the disclosure are shown. This disclosure may, however, be embodied in many different forms and should not be construed as limited to the illustrated embodiments set forth herein. Like numbers refer to like elements throughout. In describing the different embodiments of the invention illustrated in the appended drawings, specific terminology will be used for the sake of clarity. The technology, however, is not intended to be limited to the specific terms used, and it is to be understood that each specific term includes equivalents that operate in a similar manner to accomplish a similar purpose. Each of the separate descriptions of the parts of the modular pedicle screw assemblies may be used interchangeably with the other described parts.

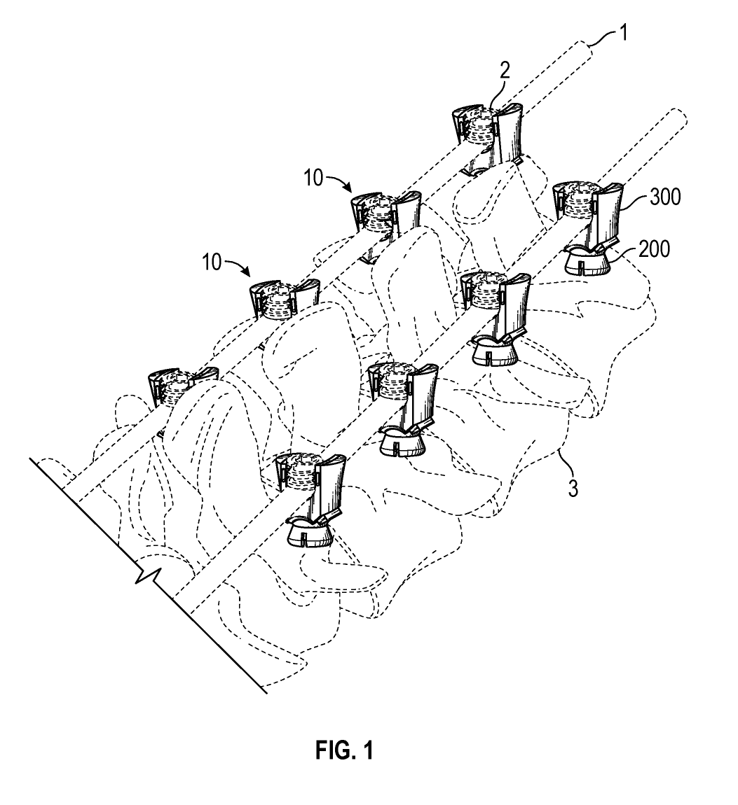

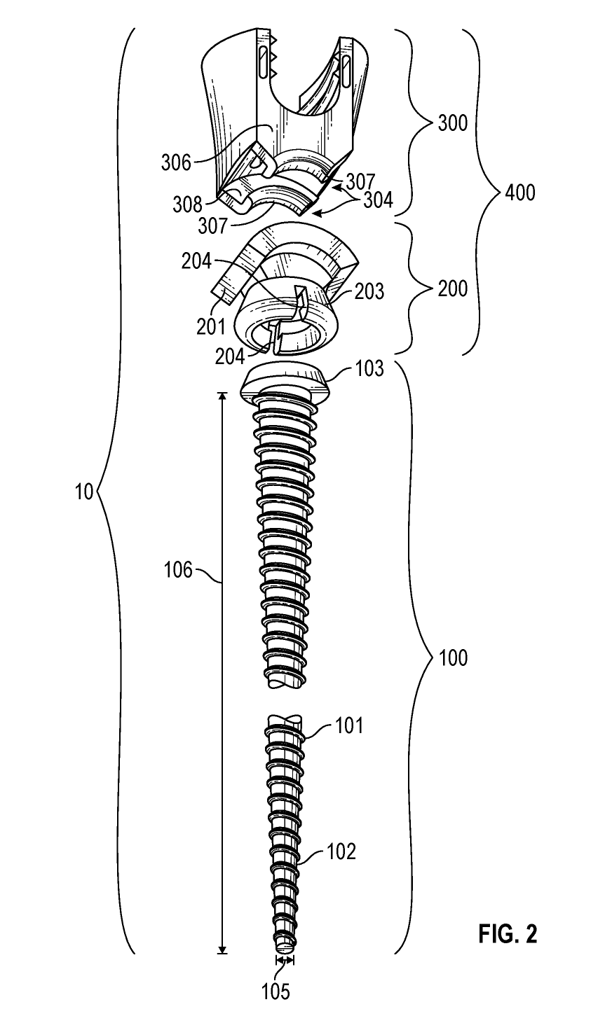

As shown in FIGS. 1-11, an embodiment according to the present disclosure provides a modular pedicle screw assembly 10 that can be comprised of a shank 100 and modular head 400. The modular head 400 can be comprised of a base 200 and a tulip head 300, as illustrated in one embodiment shown in FIG. 2. As will be discussed below with more detail, the base 200 can include a bottom portion adapted to receive a head of the shank 100, and a top portion having a slide 201. The tulip head 300 can include rails 304 adapted to move along the slide 201 of the base 200.

FIG. 1 shows a plurality of modular pedicle screw assemblies inserted into the vertebrae 3 of a spine according to an embodiment. The shanks 100 are not visible in the figure as they have been inserted into the pedicles of the vertebrae 3. The base 200 is illustrated coupled to the tulip head 300 for each modular pedicle screw assembly 10, and each series of linearly aligned modular pedicle screw assemblies 10 is shown being connected by a rod 1 in order to stabilize the spine. In addition, set screw 2 is shown inserted into the tulip head 300 to arrest movement of the rod 1 after initial alignment adjustments are made. Generally, rods 1 currently utilized in spinal surgery are made of titanium and are generally about 5-6 mm thick; however the materials and diameter can vary as necessary as long as the tulip head 300 is sized proportionally to accommodate the rod 1.

Referring to an embodiment illustrated in FIG. 2, the shank 100 can be inserted into the pedicle of the spine according to customary surgical procedures. The shank is often referred to as a screw or pedicle screw, and can be formed of any material and design suitable for insertion into the pedicle of the vertebral arch, which is the segment between the transverse process and the vertebral body. Typically, the shank can include a shaft 102 having threads 101 and a screw head 103. The screw head 103 can include a tool engagement profile, such as, for example, a straight slot for receiving a straight screw driver, a T-slot for receiving a Phillips head screw driver, a hex slot for receiving and Allen wrench, or any other appropriate profile for receiving an insertion tool. The length 106 of the shank, diameter 105 of the shank, and spacing of the screw threads 101 can vary as necessary. For example, the shank geometry can be cylindrical (constant diameter) or can be conical (increasing diameter). Generally in the surgery setting, the surgical tray can have a number of different screws that provide a selection of a variety of shank lengths and diameter combinations for use by the surgeon as necessary. The screw head 103 can be sized to fit inside the base 200 of the modular head 400.

Still referring to FIG. 2, the modular head 400 can be formed of a base 200 and a tulip head 300. The tulip head 300 can be configured to interlock with a top portion of the base 200. In an embodiment illustrated in FIG. 2, the top of base 200 can be curved and configured to couple with the complementarily curved underside and grooves of tulip head 300. This curvature can allow tulip head 300 to rotate in an arcuate motion across the top of base 200.

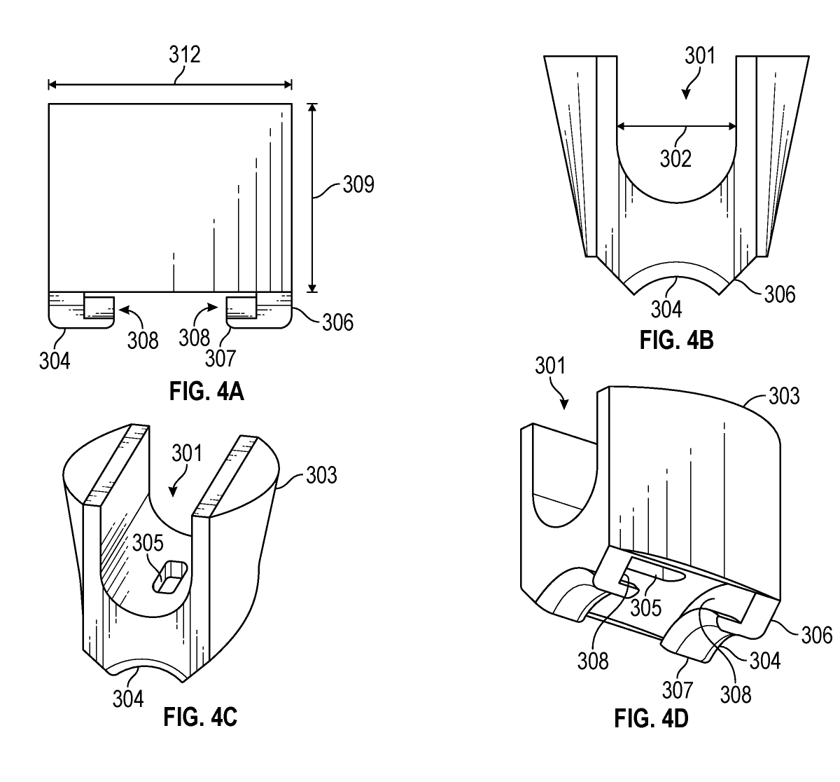

In an embodiment, tulip head 300 can be coupled with base 200 prior to coupling the base 200 to the implanted pedicle screw shank 100. For example, the pedicle screw shank 100 can be implanted in the patient to achieve desired placement. The tulip head 300 coupled to the base 200 can then be coupled to the implanted pedicle screw shank 100 to achieve a desired orientation and directionality, before receiving the rod 1. The tulip head 300 can have rails 304 formed from a side portion 306 and a bottom flange portion 307 thereof, such that the rails 304 are configured to move along the slide 201 of the base 200 as illustrated in an embodiment shown in FIG. 4. The rails can define recesses 308 configured to receive the slide 201 of the base 200.

In order to achieve coupling between the tulip head 300 and the base 200, the slide 201 can be inserted into the recesses 308. Once coupled, the tulip head 300 can be slid in an arcuate motion across the slide 201 of the base in either direction in order to achieve a desired orientation of the tulip head 300 for purposes of receiving a rod 1.

In another embodiment, the tulip head 300 can be coupled to the base 200 by snapping the tulip head 300 onto the base 200, rather than coupling the two pieces by sliding the tulip head 300 onto the base 200, as discussed above. For example, the bottom flange portion 307 of the tulip head 300 can be formed of a material, such as plastic or another suitable composite or material, which is sufficiently bendable to enable the rails 304 to expand outward as the bottom flange portion 307 is pressed down onto the top slide 201 of the base 200. Once the rails 304 have been pushed beyond the outer edges of the slide 201, the rails 304 can snap back into place, securely coupling the rails 304 to an underside of the slide 201. To facilitate the joining of the tulip head 300 and the base 200 in this embodiment, corners of the slide 201 and the rails 304 can be beveled or rounded. The rails 304 can then be slid in an arcuate motion in either direction across the slide 201 of the base in order to achieve a desired orientation of the tulip head 300 for purposes of receiving a rod 1.

In another embodiment, tulip head 300 can be coupled with base 200 after base 200 has been coupled to the implanted pedicle screw shank 100. For example, the pedicle screw shank 100 can be implanted in the patient, either with the base 200 coupled to the screw head 103 before implantation, or with the base 200 being coupled to the screw head 103 after implantation. In either example, the base 200 can be snapped onto the screw head 103 by pressing the shank receiver 203 of base 200 down onto and over the top of screw head 103. As the shank receiver 203 is pressed against the top of screw head 103, notches 204 in the perimeter of the shank receiver 203 can permit the shank receiver 203 to expand to accommodate the diameter of the screw head 103, before snapping back into place once the perimeter of the shank receiver 203 has cleared the edge of screw head 103. In some embodiments, relative movement between the base 200 and the screw head 103 can be reduced or eliminated by the provision of a locking feature. Such a locking feature can take the form of the shim 310, described above as locking motion between the tulip head and the base, or can take any other appropriate form. In one embodiment, a piece can be inserted through the base 200 substantially along the axis A_{SR}, and into contact with the screw head. This piece can be aligned with the shim so that when the shim is pushed toward the base by the rod, the shim in turn pushes the piece into contact with the screw head 103. Friction between the piece and the screw head can thereby arrest movement between the base 200 and the screw head 103.

After the base 200 has been coupled to the implanted pedicle screw head 103, the tulip head 300 can be coupled to the base 200, according to an embodiment. For example, tulip head 300 can be coupled to base 200 by sliding the bottom flange portion 307 of tulip head 300 over the slide 201 of base 200 at either end of the slide 201, as described in more detail above. In either instance, once tulip head 300 has been coupled to base 200, rails 304 of tulip head 300 can then be slid in an arcuate motion in either direction across the slide 201 of the base in order to achieve a desired orientation of the tulip head 300 for purposes of receiving a rod 1. Alternatively, in some embodiments, the tulip head 300 can be joined to the base 200 before the base 200 is coupled to the screw head 103. Such pre-assembly of the tulip head 300 and base 200 may be more efficient, depending on the circumstances of a particular operation and the positioning of the pedicle screw assemblies 10 in the spine of a patient.

In an embodiment, one or more portions of the pedicle screw assembly—including the shank 100, base 200, and tulip head 300—can be manufactured in a coupled configuration. For example, base 200 can be fabricated being coupled to screw head 103 of pedicle screw shank 100, such that a surgeon can implant the coupled screw shank 100 and base 200, without the need for coupling the two pieces either before or after implantation. In order to achieve the desired orientation of the pedicle screw shank 100 and coupled base 200, the surgeon can rotate base 200 up to 360 degrees around the screw head 103 until the desired orientation is achieved. After such desired orientation is achieved, the surgeon can couple the tulip head 300 to the base 200 and shank 100, as described in more detail above.

In another embodiment, base 200 can be fabricated being coupled to tulip head 300, such that a surgeon can couple the base 200 and tulip head 300 to the pedicle screw shank 100, either before or after implantation. In this configuration, the coupled base 200 and tulip head 300 can be coupled to the screw head 103 of pedicle screw shank 100, as described above, and can be rotated on the screw head 103 up to 360 degrees to achieve the desired orientation. Although pre-coupled to the base 200 in this embodiment, tulip head 300 can still be further slid in an arcuate motion across the slide 201 of base 200 such that additional adjustments can be made to achieve the desired orientation.

In another embodiment, each of the pedicle screw shank 100, base 200, and tulip head 300 can be fabricated being coupled together. In this example, a surgeon can implant the pedicle screw assembly in the patient, and make adjustments to the orientation after implantation. For example, the surgeon can rotate the base 200 on the screw head 103 of pedicle screw shank 100, up to 360 degrees, and can rotate the tulip head 300 in an arcuate motion across the slide 201 of base 200 until the desired orientation is achieved.

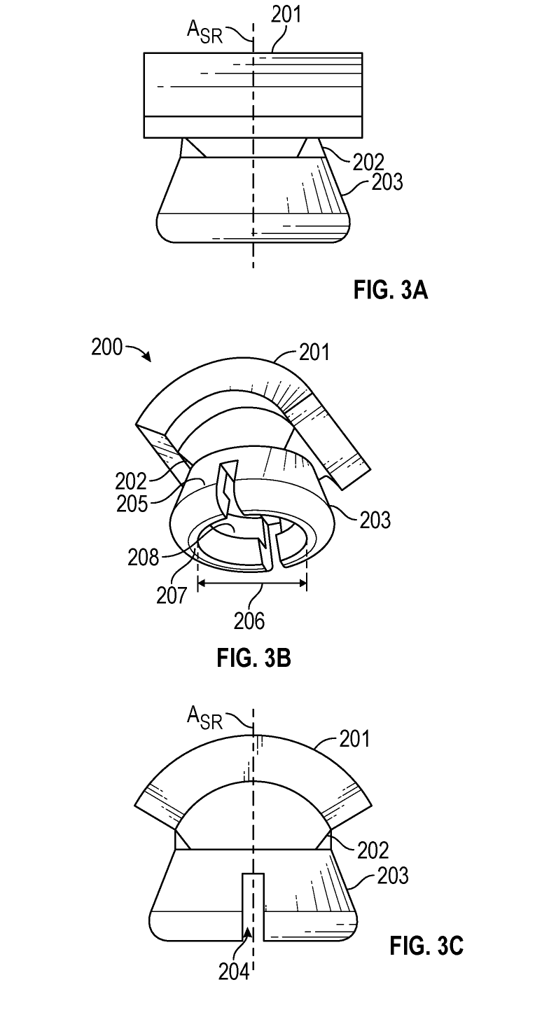

FIGS. 3A-3C illustrate a configuration of base 200 according to an embodiment of the present technology. As shown, the base 200 can be formed of three portions: the slide 201, the slide support 202, and the shank receiver 203, which can be oriented around a shank receiver axis ASR (shown in FIGS. 3A and 3C). The shank receiver 203 can be the portion of base 200 that fits over the screw head 103 of the shank 100 and mates with the screw head 103. The interior dimension 206 of the shank receiver 203 can be sized appropriately to receive the screw head 103 of the shank. Specifically, the shank receiver may include shank receiver sidewalls 205 and shank receiver ends 207. The shank receiver ends can extend radially inward toward the shank receiver axis ASR so as to define a shank receiver recess 208 (shown in FIG. 3B). The shank receiver recess 208 is configured to accept the screw head 103 and, in use, to prevent axial movement of the screw head 103 relative to the base 200.

The base 200 can provide one axis of rotation, allowing rotation up to 360 degrees about the shank 100. The base 200 can be configured to allow mating to the shank 100 via the screw head 103. The base 200 can be mated before or after the shank 100 is inserted into the pedicle of the vertebra 3. In some embodiments, the base 200 can be mated after the shank 100 is inserted so that the surgeon can use the appropriate tools for inserting the shank 100.

For example, after the shank 100 is inserted into the pedicle, the surgeon can mate the base 200 with the shank 100 by snapping the base 200 on the head 103 of the shank 100 of the pedicle screw. The base 200 can be rotated about the screw head 103 to provide proper alignment and positioning, providing the first axis of rotation. After the surgeon inserts the shank 100 into the pedicle, the surgeon can then attach the base 200 onto the screw head 103. The surgeon can then check the orientation of the shank and coupled base against other positioned shank and coupled bases to ensure that all of the respective slides 201 on the bases are properly orientated with respect to each other. For example, the slides 201 can be orientated in the same direction (such as parallel to other slides 201 coupled to a shank inserted into vertebra either superior or inferior to the vertebra where the instant shank and coupled base are installed), as is illustrated in an embodiment in FIG. 1. If the slides are not all properly orientated, the surgeon can rotate the bases about the screw head to achieve proper orientation of the slides. This capability for post-insertion rotation and orienting can provide an advantage over uniaxial screws, which may require the surgeon to back out the shank/screws from the bone in order to achieve alignment with other shanks.

In an embodiment, there can be a locking mechanism that can be installed to lock the base 200 down onto the screw head 103 of the shank 100 once the appropriate position is set. It is preferred, however, that all of the locking connections occur in one step to save on surgery time.

The shank receiver 203 also can include notches 204, as illustrated in FIGS. 3B and 3C. These notches 204 can be provided for stress relief and expansion of the shank receiver 203. When the base 200 is coupled to the screw head 103, the notches 204 can help the base 200 expand around the perimeter of the shank receiver 203 to receive the screw head 103 of shaft 100. Although two notches 204 are particularly shown in the drawings, more than two notches can be used if desired such as to accommodate bases 200 formed of different materials having different flexibilities or elasticity.

The slide support 202 of the base 200 can be disposed between the slide 201 and the shank receiver 203. The slide 201 can provide a second axis of movement. In an embodiment, the slide 201 can be the portion upon which the rails 304 of the tulip head 300 move across the slide 201 of the base, which provides the surgeon the ability to position the tulip head in alignment with other inserted modular pedicle screw assemblies. This way, if the pedicle screws were not positioned precisely in line with other pedicle screws in the pedicles inferior or superior to the instant pedicle during insertion, the surgeon can move the tulip heads 300 along the slide 201 in an arcuate motion so that all of the modular pedicle screw assemblies are in alignment to receive the rod. This configuration alleviates the problem of the rod exerting excessive force on any one of the misaligned assemblies, and accordingly reduces instances of breakage of such assemblies or undue stresses on the vertebrae of the patient.

The slide 201 optionally has “stops” (not shown), which can include either protrusions or recesses to arrest the movement of the rails of the tulip head along the slide 201. In addition, after the tulip head 300 is properly positioned along the slide 201, the tulip head 300 can be locked down by a stop or locking mechanism to arrest relative movement between the tulip head 300 and the slide 201.

Referring now to FIGS. 4A-4D, the tulip head 300 can have rails 304 formed from a side portion 306 and a bottom flange portion 307 that define recesses 308 configured to receive the slide 201 of the base 200. This configuration can provide the ability of the tulip head 300 to move along the slide 201 and be locked into any position along the slide 201. This configuration can also provide a second axis of movement in the modular pedicle screw assembly.

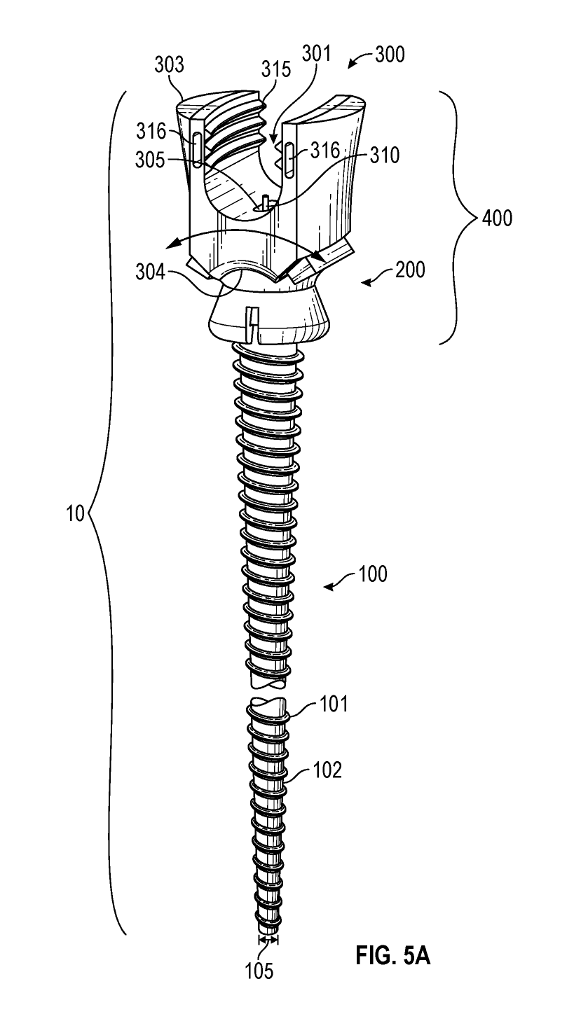

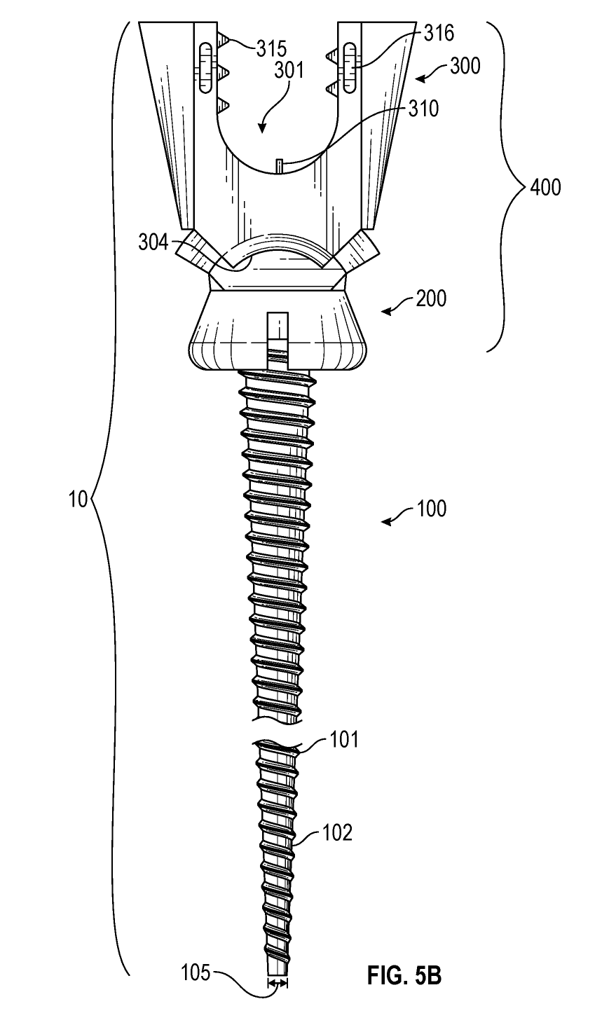

The tulip head 300 also can have a through-hole 305. This through-hole 305 can be any shape (such as, but not limited to, elongate, oval, round, etc.) and any dimension necessary. A shim 310 (shown in FIGS. 5A and 5B) can be located within the through-hole 305. The shim 310 can be sized to be taller than the depth of the through-hole 305 so that the uppermost portion of the shim 310 extends into the U-shaped channel 301 of tulip head 300. In an embodiment, when the rod 1 is placed into the U-shaped channel 301 of the tulip head 300, the rod 1 can contact the shim 310 first and then contact the bottom surface of the U shaped channel 301 of the tulip head 300. As the rod 1 is tightened down with a set screw 2, the rod 1 can push the shim 310 down and apply a significant friction on the slide 201, thereby locking the tulip head 300 in a single orientation in place on the base 200. Although the shim 310 is shown to be cylindrical in shape, it can be any appropriate shape. In addition, the shim 310 can be coupled with the tulip head 300 so that it will not fall off or become disengaged from the tulip head 300 during assembly or when the tulip head 300 is handled separately by a surgeon. In one example embodiment, the through-hole 305 can be threaded to receive threads of a threaded locking mechanism.

The tulip head 300 can be configured to include hips 303, which can provide support and structure around the U-shaped channel 301. Although shown in certain figures to be conical in shape, the tulip head 300 can be any appropriate shape, including, for example, cylindrical. The U-shaped channel 301 can be configured to receive a rod 1, which can be a stabilizing member used in spinal surgery to stabilize the spine. The width 302 of the U-shaped channel 301 can be sized to receive a rod 1 that is customarily used in the spinal stabilization surgery. Typically the rod can be about 5-6 mm in diameter and is can be composed of titanium or any other appropriate material.

The tulip head 300 can be configured to include rails 304 that are able to move along the slide 201 of the base 200. This configuration can provide flexible and accurate placement and orientation so that when the surgeon installs the rod 1 in the U-shaped channel 301 after inserting the shank 100 into the pedicle, the tulip head 300 can be moved along the slide 201 to allow for proper alignment of the rod 1. In addition, as noted earlier, the base 200 can be rotated to be in appropriate orientation and alignment so that the surgeon does not need to back out the shank from the pedicle for proper positioning.

The dimensions of the overall tulip head 300 and its various components (i.e., the width 302 of the U-shaped channel 301, the width 312 and height 309 of the tulip head 300) can be sized appropriately for use in standard medical protocols using pedicle screws. The side of the rails 306 and the bottom flange 307 of the rails can be sized to allow movement along the slide 201 of the base 200. In addition, the rails 306 and bottom flange 307 can be sized to have sufficient strength to function without breaking during an expected life once implanted within a patient. In the drawings, principally for the sake of clarity, all side walls intersecting orthogonally are shown to have 90 degree corners (e.g. the inner walls of the rails 308). During the manufacturing process, however, such intersections may have a slight rounding due to the limitations of the machines producing the devices.

As illustrated in FIGS. 5A and 5B, in some embodiments the tulip head 300 can have threads 315 within the U-shaped channel 301 to mate with threads of a set screw 2 (see FIG. 1), which is inserted on top of the rod 1 to hold the rod 1 in place once inserted into the U-shaped channel 301. The tulip head 300 can optionally have recesses 316 for grabbing the tulip head 300 with a tool during surgery.

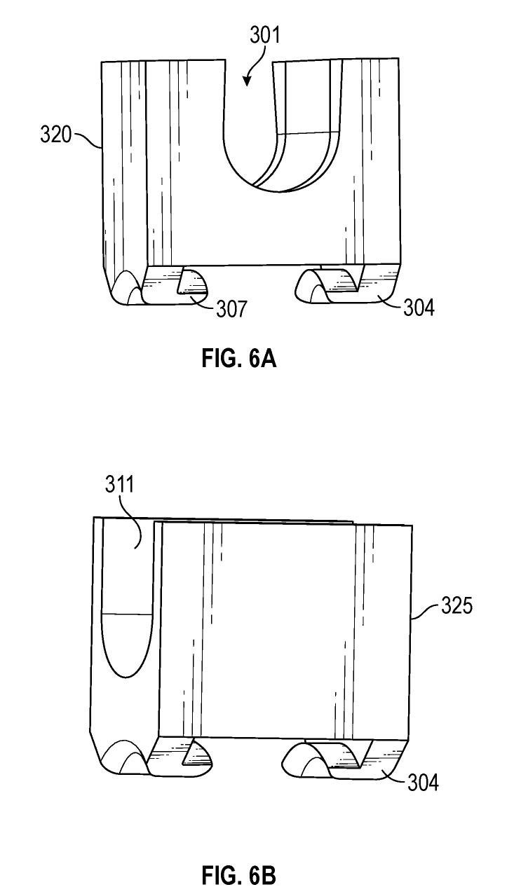

According to some embodiments of the present technology, tulip heads 300 can be configured such that the U-shaped channel 301 runs parallel with the rails 304, as shown in an embodiment illustrated in FIG. 6A, or the U-shaped channel 311 can be configured to run perpendicular to the rails 304, as shown in an embodiment illustrated in FIG. 6B. A surgeon may utilize both of these orientated tulip heads 300 selectively as needed to allow placement of the rod in order to reduce the amount of stress on the rod and the modular pedicle screw assembly.

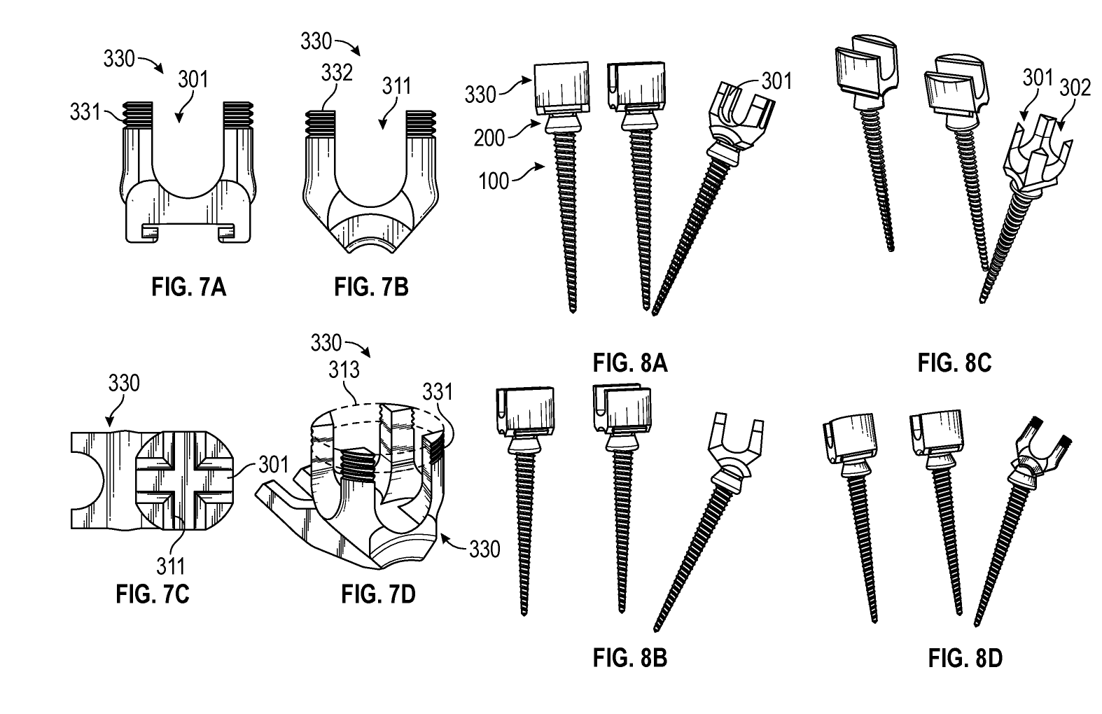

Some embodiments may also include a dual U-channel tulip head 330, as illustrated in an embodiment shown in FIGS. 7A-7D. In such embodiments, there can be two U-shaped channels 301 and 311. One U-shaped channel 301 can be configured to run parallel with the rails 304, while the other U-shaped channel 311 can be configured to run perpendicular to the rails 304. FIG. 7C also shows a top view of the dual U-shaped channel tulip head with the four prongs, and FIG. 7D shows a perspective view of the dual U-shaped channel tulip head. In this embodiment, instead of using a set screw 2 that is placed and threaded inside the tulip head 300 to fix the rod 1, a ring 313 can be placed on the tulip head 300 to encompass the four prongs 332. The ring 313 can engage threads 331 positioned on the outside surface of the prongs 332. The ring can be threaded outside the circumference of the tulip head 300 on the perimeter threads 331 to provide strength and stability to the tulip head 300 and to prevent the tulip head 300 from spreading open under excessive loading. FIGS. 8A-8D provide four depictions of a dual U-channel tulip head 300 rotated to accommodate different orientations to receive the rod, according to an embodiment of the present technology.

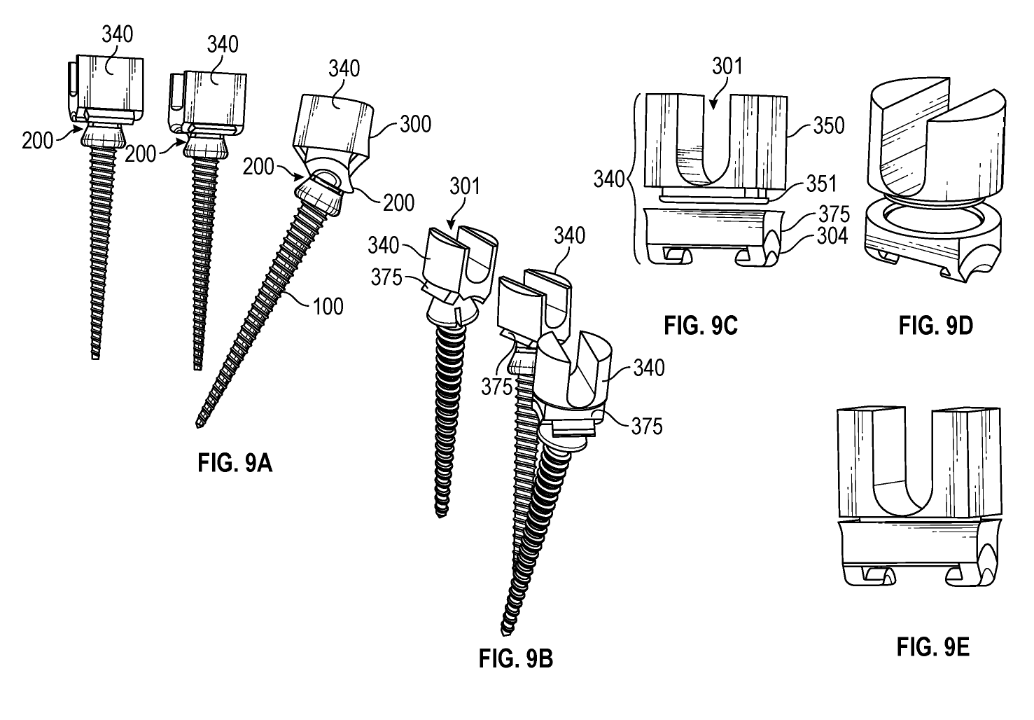

FIGS. 9A-9E show a two-part, rotatable tulip head 340 having an upper section 350 including the U-shaped channel 301, and a lower section 375 including the rails 304, according to an embodiment. This embodiment allows the upper section 350 to rotate about the lower section 375, where the upper section capable of rotating up to 360 degrees independently of the lower section 375. Upper section 350 and lower 375 can be mated together, for example, by screwing the upper section 350 into a threaded portion of the lower section 375, or by any other appropriate means. FIGS. 9C-9E show the lower section 375 (i.e., the female end) configured to receive the upper section 350 (i.e., the male end 351). In another embodiment, the upper section 350 can be configured to receive the lower section 375 (i.e. the lower section 375 can be configured to have the male connector and the upper section 350 can be configured to be the female receiver).

FIGS. 9A and 9B show a side perspective view of three modular pedicle screw assemblies, where one of the assemblies includes a two-part, rotatable tulip head 340, according to an embodiment of the present technology. FIG. 9A shows that the two part, rotatable tulip head 340 can be positioned along the base 200. FIG. 9B shows the two-part, rotatable tulip head 340 rotated about the lower section 375. The multiple axes of movement provide improved flexibility to the surgeon for aligning the U-shaped channel 301 to any desired orientation and alignment.

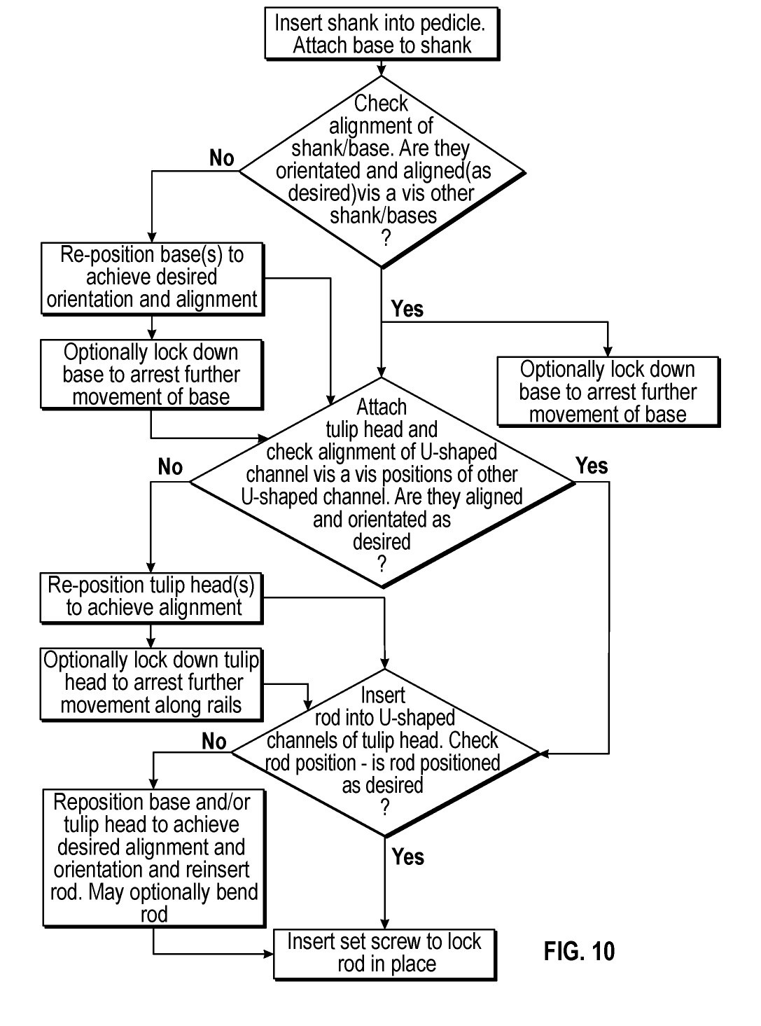

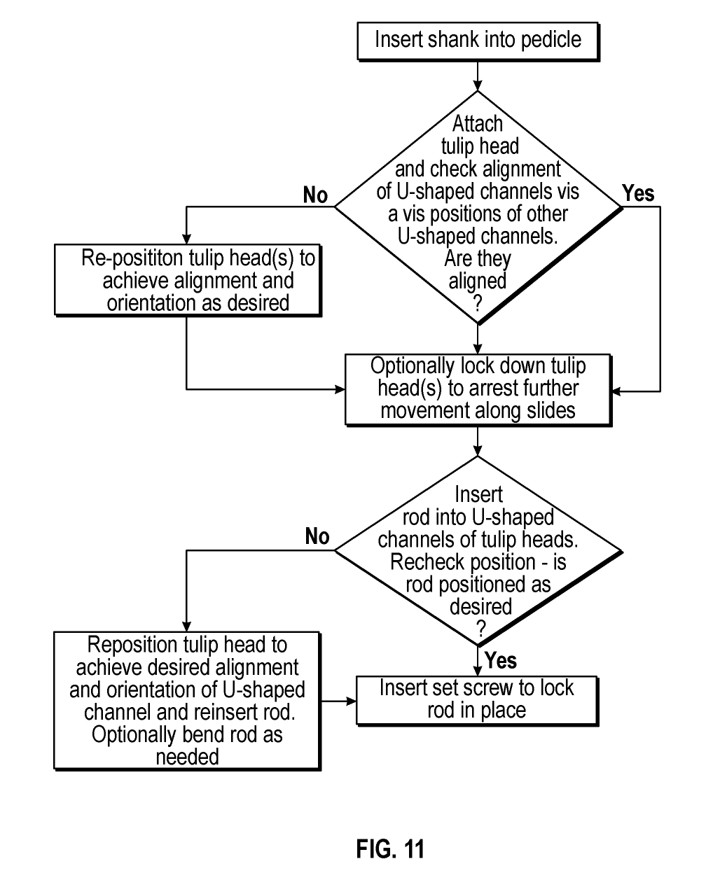

FIGS. 10 and 11 are flowcharts illustrating a method of achieving alignment of the U-shaped channels in the modular pedicle screw assemblies discussed above to allow placement of the rods within the aligned an oriented U-shaped channels. This ensures that the rod does not exert undesired pressure upon any of the assemblies, which would occur if they were not in the desired alignment or orientation.

As shown in FIG. 10, a surgeon can insert a pedicle screw shank into a pedicle of a patient's vertebrae. After the surgeon inserts the shank into the pedicle, the surgeon can attach the base onto the screw head. The surgeon can then check the alignment and orientation of the shank and coupled base against other implanted and positioned shank and base assemblies to ensure that all of the slides on the bases are properly orientated or positioned and aligned as desired, (as shown in FIG. 1). If the slides are not all properly oriented or positioned, the surgeon can reposition the bases by rotating the bases about the screw head to achieve proper orientation of the slides. The surgeon can then optionally install a locking mechanism to ensure the base does not move after alignment.

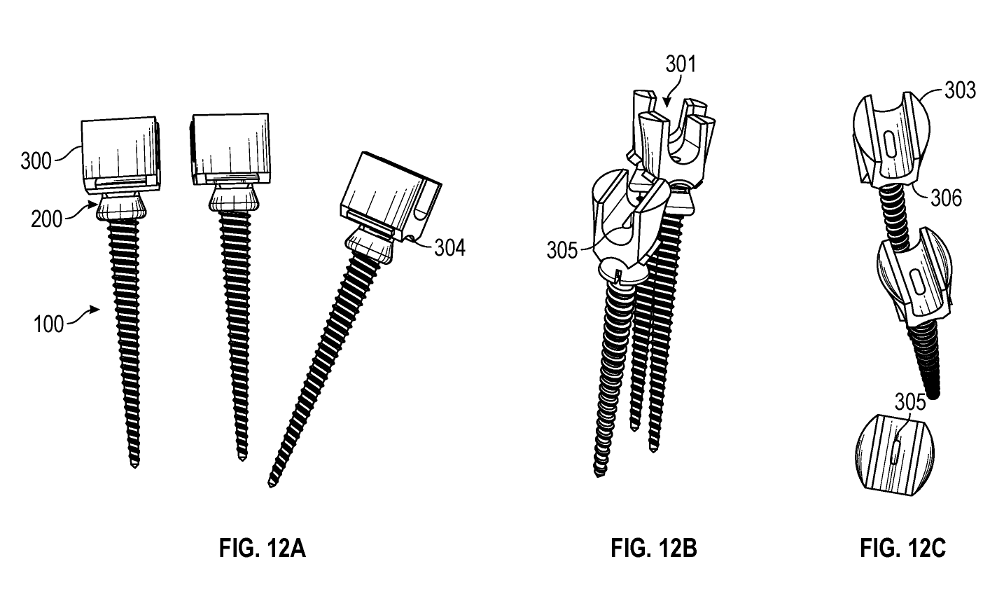

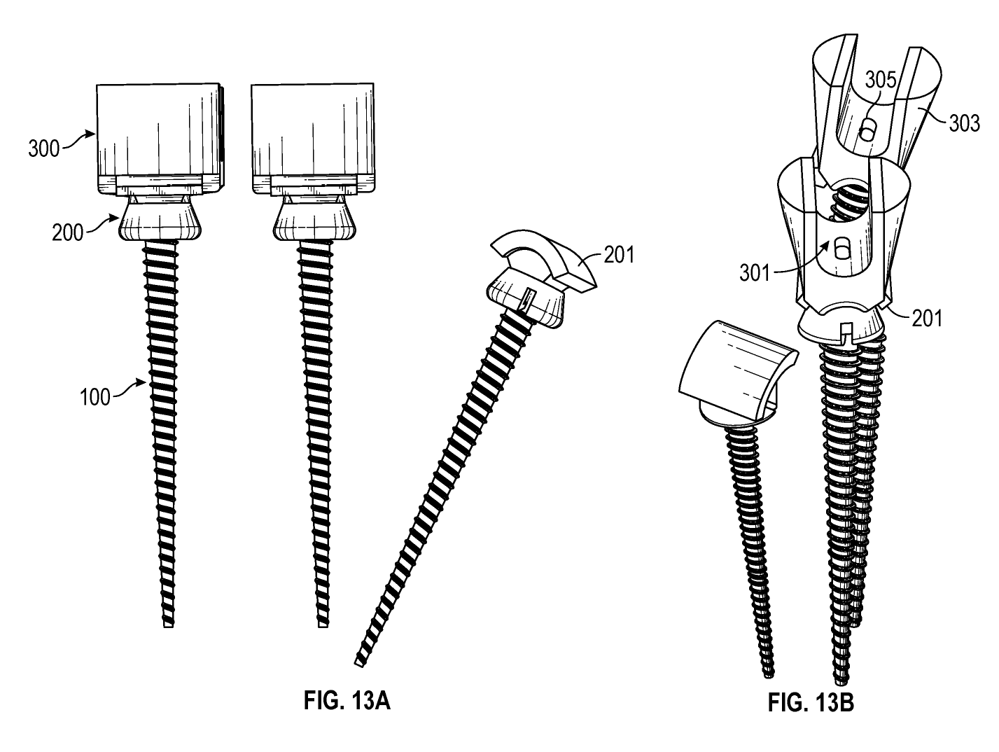

Pedicle screws are often not aligned perfectly on a single plane and are usually tilted and offset with respect to each other, as illustrated, for example, in the embodiments shown in FIGS. 12A-12C and FIGS. 13A-13B. For example, the surgeon may need to rotate one of the modular pedicle screw assemblies relative to a neighboring modular pedicle screw assembly. FIG. 13B shows an embodiment in which one modular pedicle screw assembly is rotated so the base can be about 90 degrees offset from the other modular pedicle screw assembly. Also, because of the curvature of the spine, the screws can be positioned in such a manner that causes them not to be aligned on a single plane, as illustrated in FIGS. 12A, 12B, and 13A. Such deviation in the orientation of presently available pedicle screw assemblies is problematic, since the titanium rods may only be bent and curved so much to accommodate the necessary deviation. The present disclosure provides solutions to this problem.

For instance, the surgeon can utilize a mixture of two different tulip heads: one with a parallel orientated U-shaped channel 320 and the other with a perpendicular orientated U-shaped channel 350, as illustrated in an embodiment described above and shown in FIGS. 6A and 6B.

In another embodiment, the surgeon can utilize a dual U-shaped channel tulip head 330. As described above, this dual U-shaped channel tulip head 330 can have dual U-shaped channels 301, 311—one perpendicular and one parallel to the rails—as described above and shown in FIGS. 7A-8D. FIGS. 8A-8D provide four depictions of a dual U-shaped channel tulip head 330 rotated to accommodate different orientations that could be required to receive the rod. When using a dual U-shaped channel tulip head 330, preferably a ring 313 can be placed (via threading or otherwise) around the perimeter of the tulip head to ensure that the walls of the tulip head do not spread open under excessive loading. It is also possible to use a set screw 2 that is threaded inside the tulip head (such as that shown in FIG. 1) to fix the rod in place.

In another embodiment, the surgeon can employ the use of a two-part, rotatable tulip head 340, such as that described above and shown in FIGS. 9A-9E, which can be configured to rotate up to 360 degrees on the base to provide ultimate flexibility to the surgeon for aligning the U-shaped channel to any desired orientation.

A surgeon could then use any of the tulip heads described herein together (for example, use a dual U-shaped channel tulip head 330 with the two-part, rotatable tulip head 340) to provide flexibility in positioning the pedicle screw assemblies to accommodate the rod and to provide many positions and orientations without requiring bending and curving of the rod.

Referring again to the method illustrated in FIG. 10, after the shank and base are installed, the surgeon can install the tulip head onto the base 200. The surgeon can then check that the U-shaped channels in the tulip heads are in a desired alignment and orientation relative to one another. If the tulip heads are not in the desired orientation, they may be repositioned to achieve the desired orientation. For example, the entire tulip head may be rotated in an arcuate motion across the slide of the base, for example as illustrated by the arrows in FIG. 5A, or a top portion of the tulip head may be circularly rotated around a bottom portion of the tulip head in another embodiment, or a combination thereof.

Once the desired alignment and orientation is achieved, the surgeon then can employ a locking mechanism to arrest movement of the tulip head along the slide of the base. The surgeon can also install the rod into the U-shaped channels of the tulip heads. If desired, the surgeon can recheck the position of the rod and make any necessary adjustments of the tulip head and/or base to ensure proper alignment and orientation. The rod can also be bent as necessary. Once satisfied with the position of the rod, the surgeon can then insert and tighten down a set screw on the rod to hold the rod in place within the U-shaped channel of the tulip head.

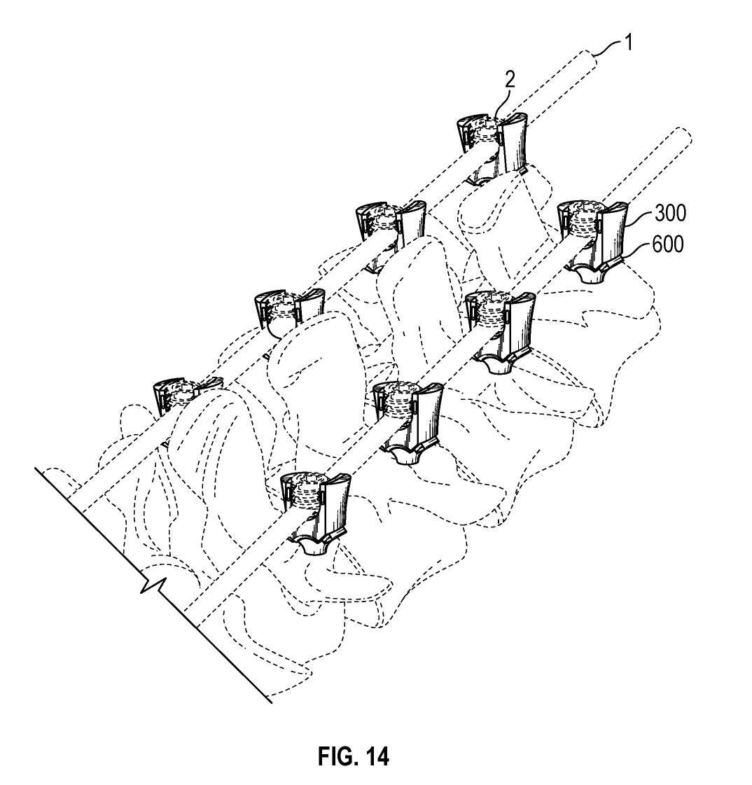

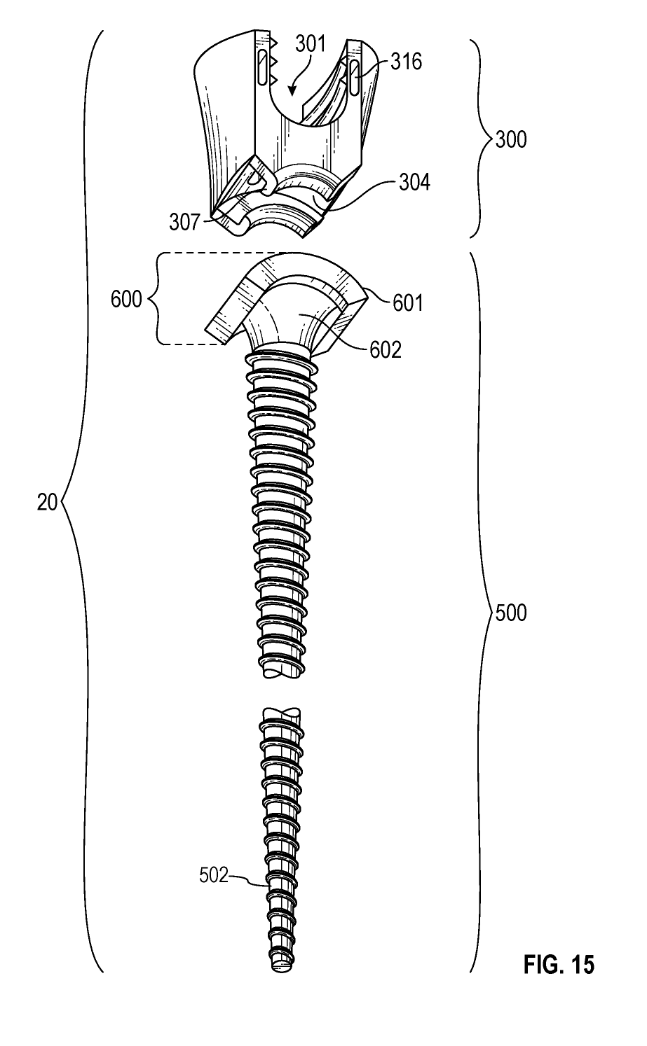

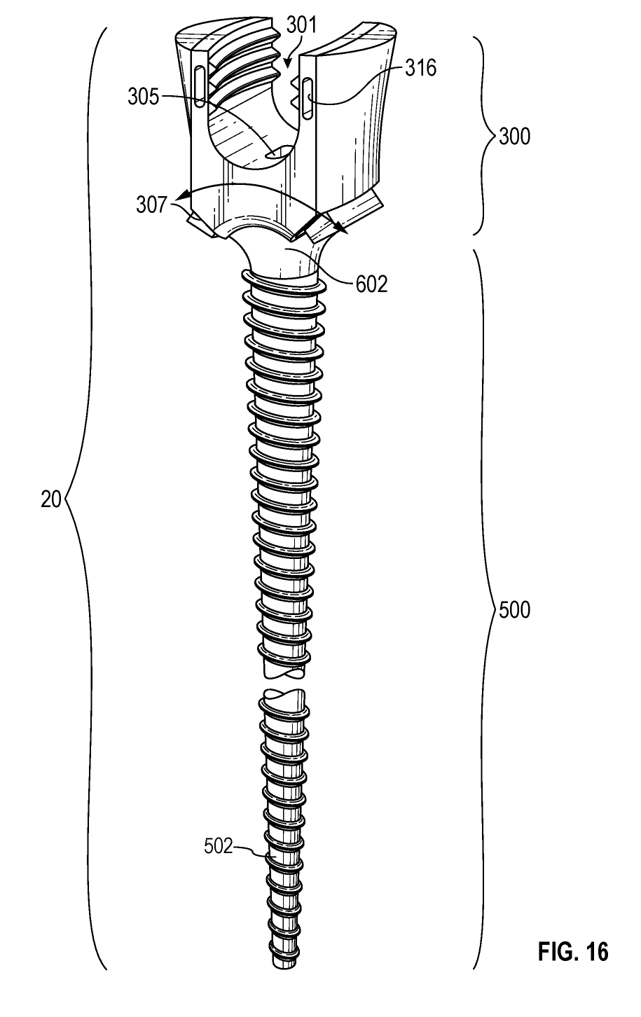

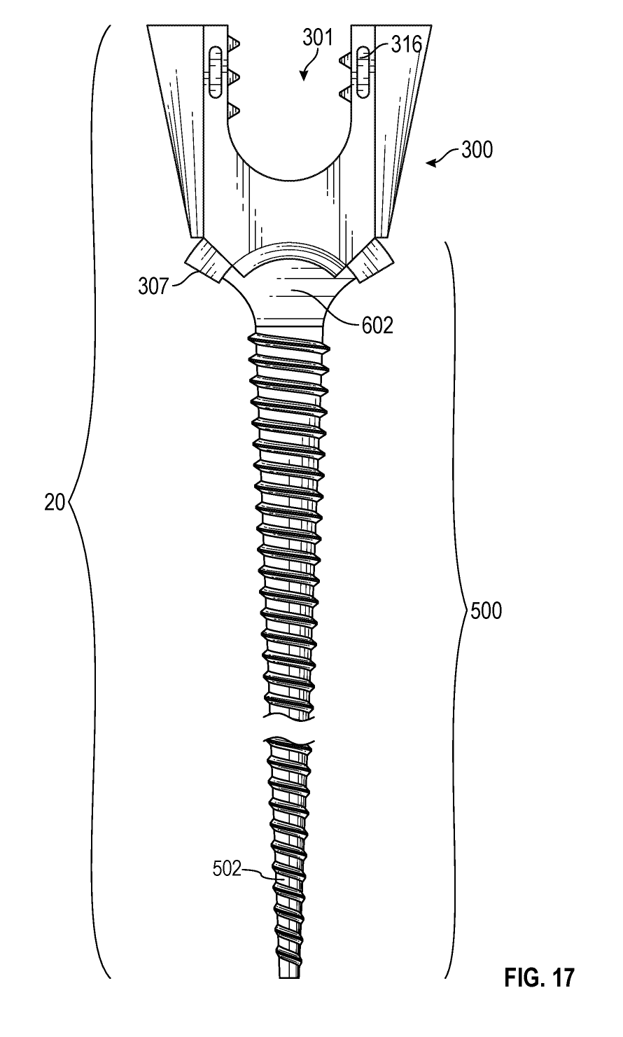

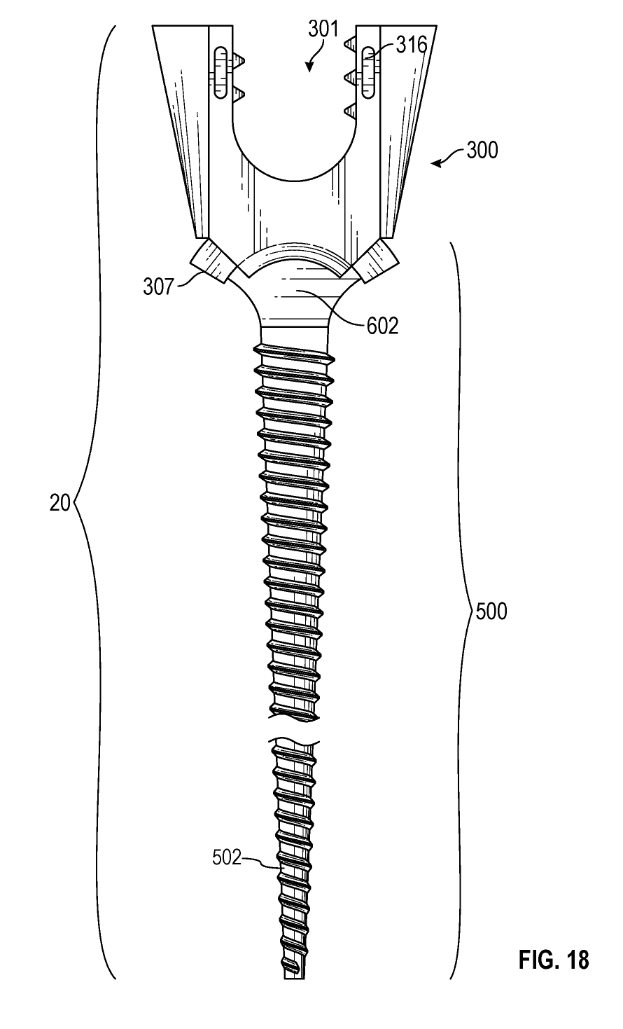

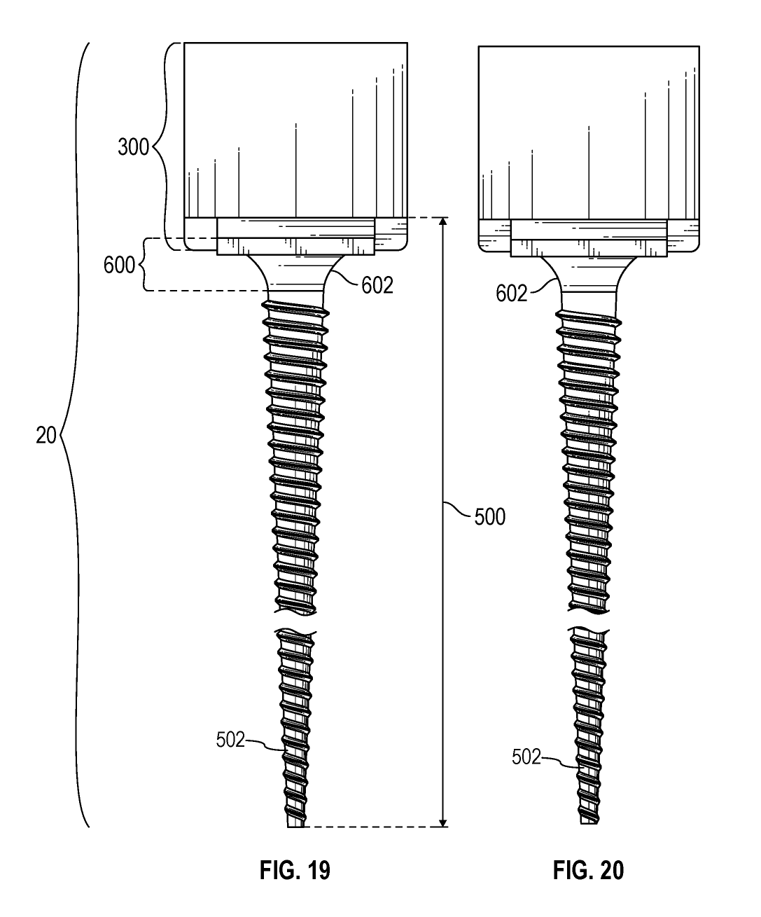

As shown in FIGS. 13-20, and in particular in FIG. 14, in another embodiment, the modular pedicle screw assembly 20 can include a pedicle screw 500 having an arched head 600 and a tulip head 300. The tulip head 300 can be any of the tulip heads as described above, including parallel U-shaped channel tulip heads 320, perpendicular U-shaped channel tulip heads 325, dual U-shaped tulip heads 330, and two-part, rotatable tulip heads 340. In the illustrated embodiment, the pedicle screw 500 can have an arched head 600 integrally formed thereon, instead of having a typical, flat-topped screw head configured to couple to a separate arched base. The arched head 600 provides the slide 601 upon which the rails 304 of the tulip head 300 can slide, for example as illustrated by the arrows in FIG. 16, to allow the surgeon to fine tune the positioning and alignment for proper positioning for receiving the rod 1 into the U-shaped channel 301 of the tulip head 300. The arched head has the slide portion 601 and the slide support 602.

The arched configuration seen in the arched head 600, as well as in the base 200, can provide the ability to achieve a desired orientation, given the natural curvatures of the spine and the different sizes of vertebrae. When pedicle screws are placed in pedicles, they may be orientated at slightly different angles, such that the rods connecting these screws may be slightly bent or curved before placement.

Referring back to FIG. 11, there is shown another method for achieving a desired alignment and orientation of the U-shaped channels using embodiments of modular pedicle screw assemblies discussed above with regard to FIGS. 13-21. The method of FIG. 11 allows placement of the rods within the aligned U-shaped channels to ensure that the rod does not exert undue or unwanted pressure upon any of the assemblies, which would occur if they were not in the desired alignment and orientation.

As illustrated in FIG. 11, the surgeon can insert the shank into the pedicle and position the shank so that the slides are in the desired orientation and alignment relative to other positioned shanks. This ensures that all of the slides are orientated as desired, as illustrated in the embodiment shown in FIG. 13. The surgeon can then install the desired tulip head onto the arched head. The surgeon can next check that the U-shaped channels in the tulip heads are in the desired alignment and orientation with other U-shaped channels. Once the desired alignment and orientation is achieved, the surgeon can, if desired, employ a locking mechanism to arrest movement of the tulip head along the slide of the arched head. The surgeon can then install the rod into the U-shaped channels of the tulip heads. If desired, the surgeon can check the position of the rod and make any necessary adjustments to the tulip head (as well as bend the rod if necessary) to ensure the desired position (alignment and orientation) of the rod. Once satisfied with the position of the rod, the surgeon can then insert and tighten down a set screw over the rod to hold the rod in place within the U-shaped channel of the tulip head.

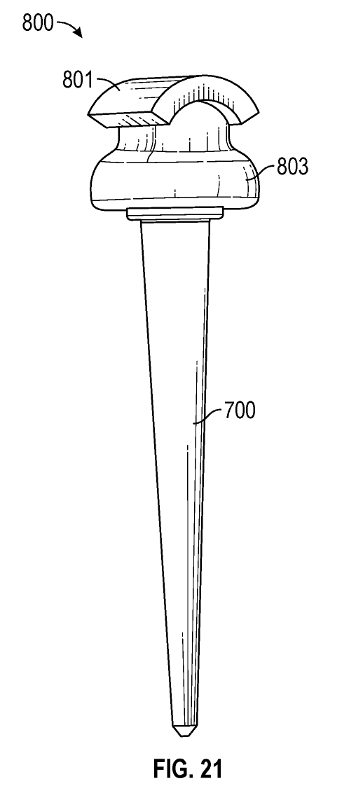

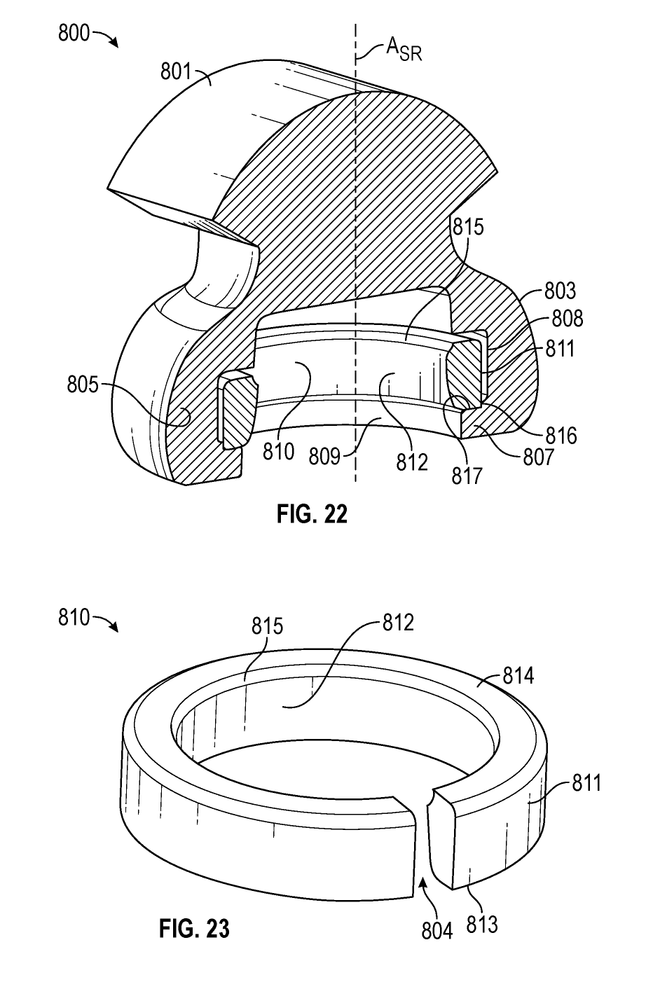

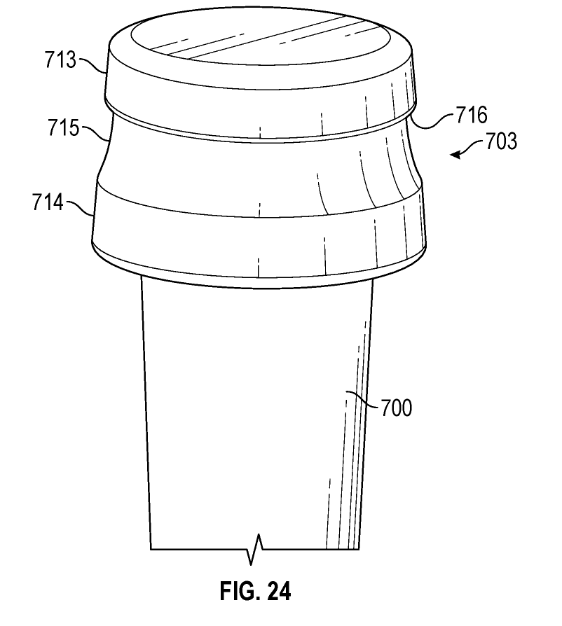

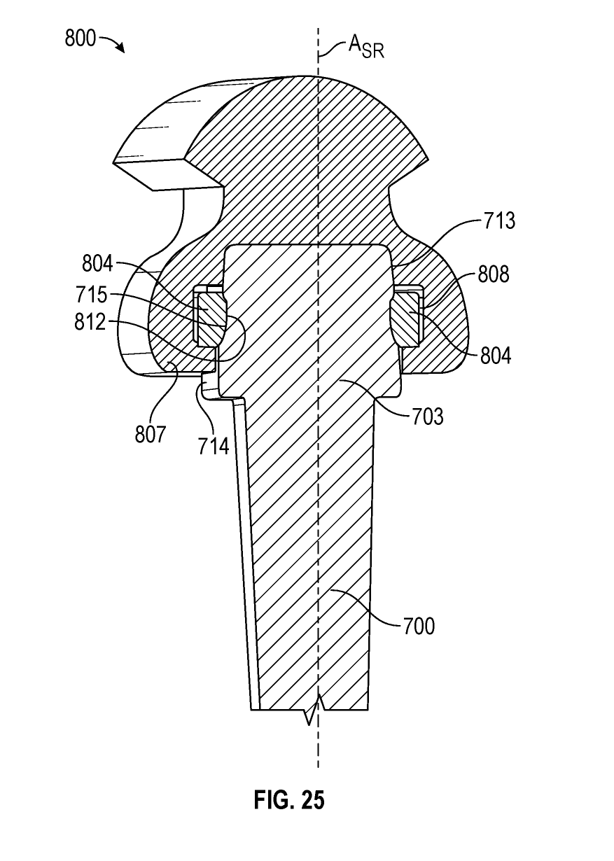

In FIG. 21 there is shown yet another embodiment of the present technology, including a shank 700 having a screw head 703 (shown in FIGS. 24 and 25). As in the above-described embodiments, the screw head 703 is configured to engage a base 800. The screw head 703 can include a tool engagement profile, such as, for example, a straight slot for receiving a straight screw driver, a T-slot for receiving a Phillips head screw driver, a hex slot for receiving and Allen wrench, or any other appropriate profile for receiving an insertion tool. The base 800 can include a slide 801 for sliding connection with a tulip head, as described above, and a shank receiver 803. The shank receiver 803 can be substantially symmetrical about the shank receiver axis ASR of the base 800 (shown in FIG. 22). In some embodiments, relative movement between the base 800 and the screw head 703 can be reduced or eliminated by the provision of a locking feature. Such a locking feature can take the form of the shim 310, described above as locking motion between the tulip head and the base, or can take any other appropriate form. In one embodiment, a piece can be inserted through the base 800 substantially along the axis ASR, and into contact with the screw head 703. This piece can be aligned with the shim so that when the shim is pushed toward the base by the rod, the shim in turn pushes the piece into contact with the screw head 703. Friction between the piece and the screw head can thereby arrest movement between the base 800 and the screw head 703.

As shown in FIG. 22, the shank receiver 803 can include shank receiver sidewalls 805, oriented substantially parallel to the shank receiver axis ASR, and shank receiver ends 807, attached to ends of the shank receiver sidewalls, and oriented substantially perpendicular to the shank receiver axis ASR. The shank receiver ends 807 may extend radially inward toward the shank receiver axis, thereby defining a shank receiver recess 808. The shank receiver recess 808 includes a recess shoulder 816 in a corner thereof adjacent a first end 817 of the shank receiver recess 808. In some embodiments, the shank receiver ends 807 are designed to circumscribe the screw head 703, and have an inner circumference 809 large enough to allow passage of the screw head 703 into the shank receiver 803 such that the shank receiver 803 substantially circumscribes the screw head 703.

Referring now to FIGS. 22 and 23, there is shown a ring 810 configured for positioning in the shank receiver recess 808. The ring 810 is substantially circular, and has an outer surface 811 and an inner contoured surface 812. The inner contoured surface tapers gradually from a first end 813 of the ring 810, and abruptly from a second end 814 of the ring 810. The abrupt nature of the taper at the second end 814 of the ring 801 forms a step 815 in the ring 810. The ring 810 can also include a ring notch 804, which allows the ring 810 to flex, so that the circumference of the ring can expand and contract. The ring can be composed of a material with sufficient elasticity that the ring returns to a rest circumference after expansion once the force is removed. In some embodiments, the ring may be composed of titanium, steel, or any other suitable material. The ring could similarly include varied geometry, such as a design that allows for increased flexibility of the ring.

FIG. 24 shows a screw head 703 according to an embodiment of the present technology. The screw head 703 includes an external contour with first and second flat portions 713, 714, and a depressed curved section 715. The depressed curved section 715 of the screw head 703 tapers gradually from the second flat surface 714, and abruptly from the first flat surface 713. The abrupt nature of the taper near the first flat surface 713 forms a shoulder 716 in the screw head 703. The depressed curved section 715 of the screw head 703 is configured to substantially align with the inner contoured surface 812 of the ring 810, so that when the depressed curved section 715 of the screw head 703 is positioned adjacent the inner contoured surface 812 of the ring 810, the surfaces mate.

FIG. 25 shows a shank 700, screw head 703, base 800, and ring 810 in a made up assembly. During assembly, which may be accomplished during surgery, such as when the shank 700 is inserted in to the spine of a patient, or pre-surgery, before the shank 700 is inserted, the screw head 703 is inserted into the shank receiver 803 of the base 800. Because the inner circumference of the shank receiver ends 807 is slightly larger than the diameter of the screw head 703, the screw head 703 is able to pass the shank receiver ends 807 without resistance from the shank receiver ends 807 themselves.

During assembly, the ring 810 is positioned within the shank receiver recess 808, and is configured so that the inner contoured surface 812 of the ring 810 extends radially inward from the circumference of the shank receiver ends 807 toward the shank receiver axis ASR. As the screw head 703 passes into the shank receiver 803, it contacts the ring 810 and exerts an upward and a radial force on the ring 810. The upward force pushes the ring away from the first end 817 of the shank receiver recess 808 and out of contact with the recess shoulder 816. The radial force simultaneously expands the ring to allow passage of the first flat portion 713 of the screw head 703 through the ring 204. Once the first flat portion 713 of the screw head 703 passes the ring 810, the depressed curved section 715 of the screw head 703 aligns with the inner contoured surface 812 of the ring 810. Upon alignment of these features, the elasticity of the ring 810 causes the ring 810 to contract so that the inner contoured surface 812 engages the depressed curved section 715 of the screw head 703. Simultaneously, the shoulder 716 of the screw head 703 engages the step 815 of the ring 810, thereby locking the screw head 703 in place within the shank receiver 803.

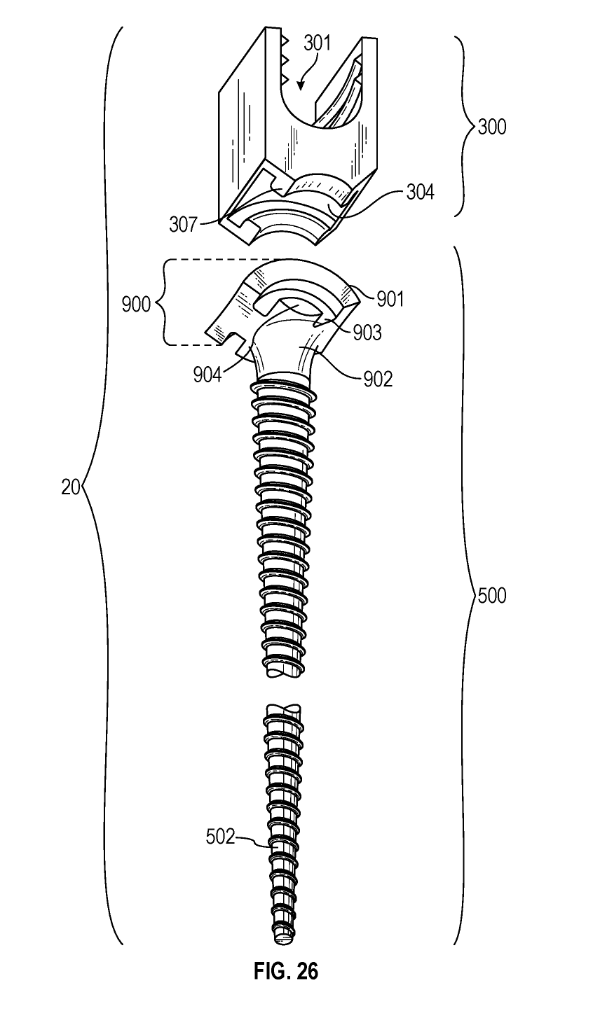

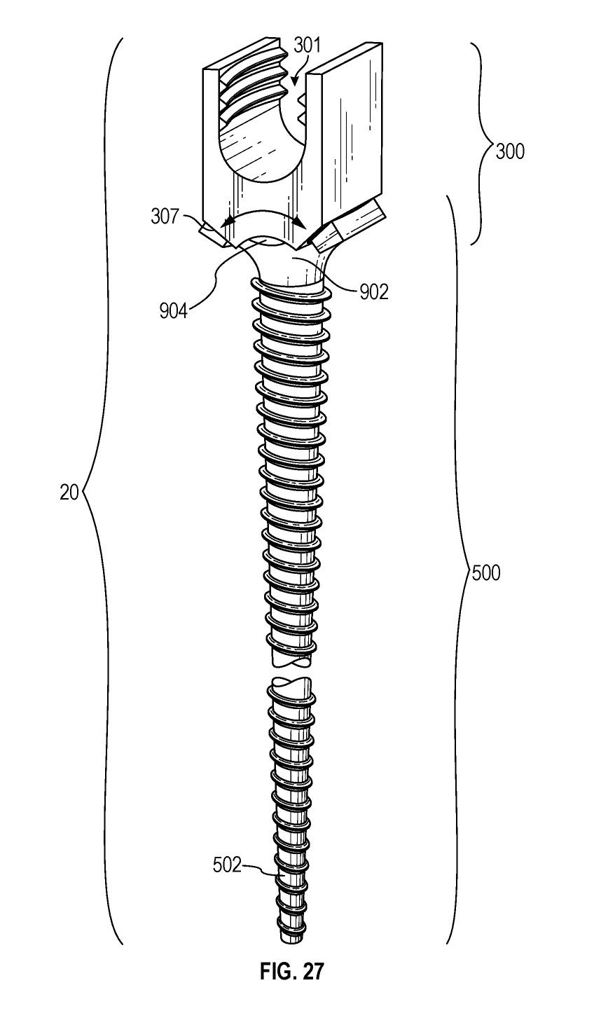

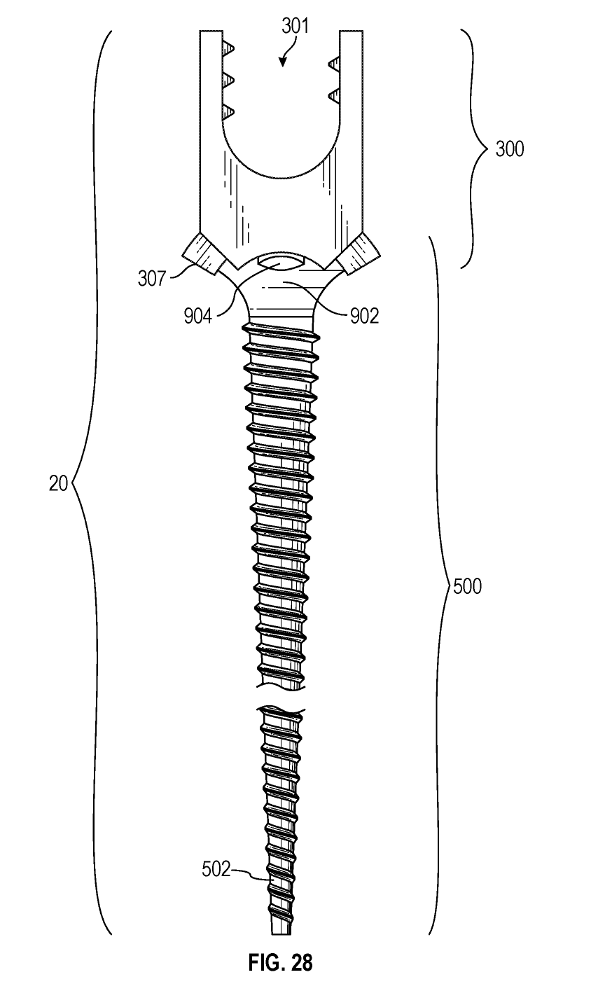

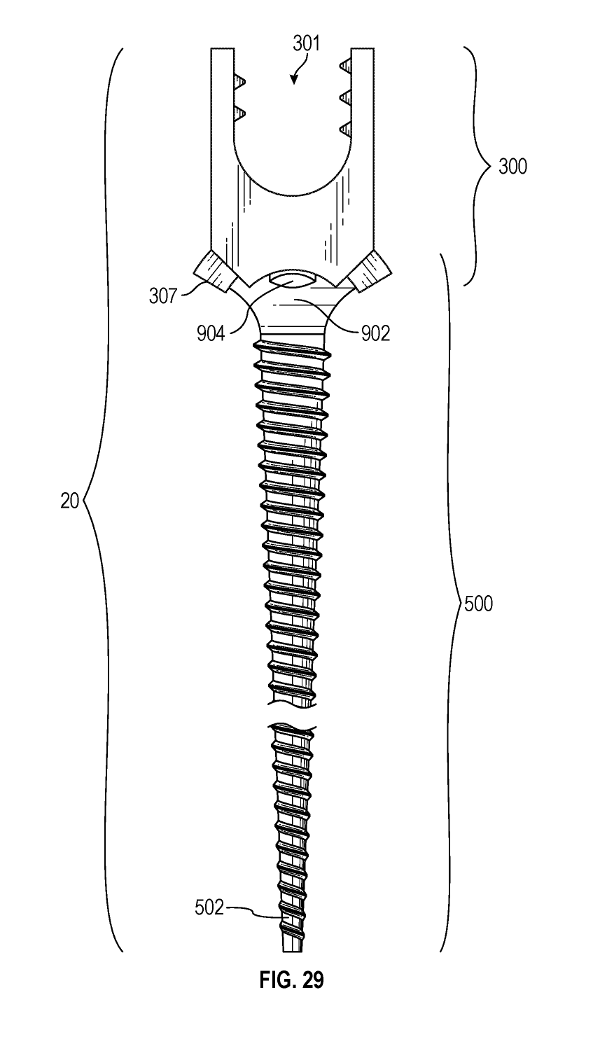

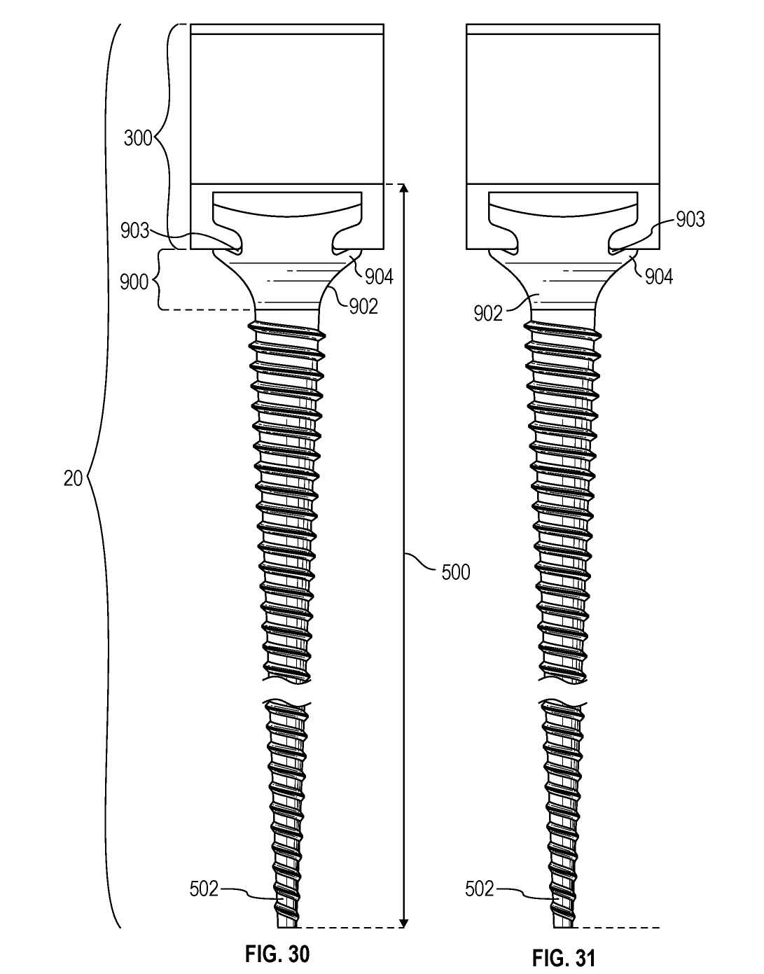

FIGS. 26-32 illustrate another embodiment of a pedicle screw and a modular pedicle screw assembly. As shown, the modular pedicle screw assembly 20 can include a pedicle screw 500 having an arched head 900 and a tulip head 300. The tulip head 300 can be any of the tulip heads as described above, including parallel U-shaped channel tulip heads 320, perpendicular U-shaped channel tulip heads 325, dual U-shaped tulip heads 330, and two-part, rotatable tulip heads 340. In the illustrated embodiment, the pedicle screw 500 can have an arched head 900 integrally formed thereon, instead of having a typical, flat-topped screw head configured to couple to a separate arched base. The arched head 900 provides the slide 901 upon which the rails 304 of the tulip head 300 can slide to allow the surgeon to fine tune the positioning and alignment for proper positioning for receiving the rod 1 into the U-shaped channel 301 of the tulip head 300. In particular, the curvature of the arched head 900 can allow the tulip head 300 to rotate in an arcuate motion across the top of the arched head 900 of the pedicle screw, as illustrated with the arrows shown in FIG. 27, for example, for purposes of receiving the rod 1. The arched head has the slide portion 901 and the slide support 902, where the slide support 902 can also be referred to as the screw neck.

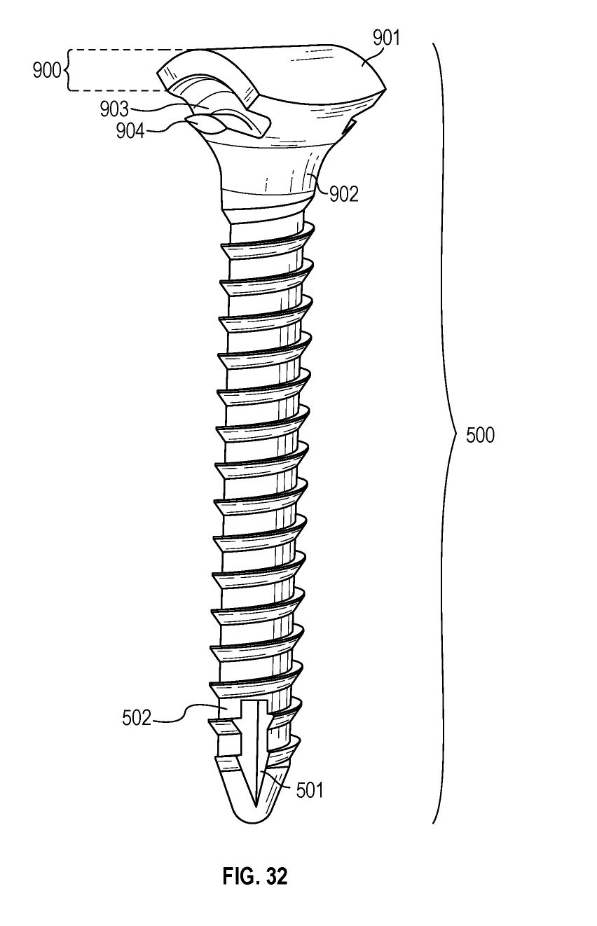

In some embodiments, for example as illustrated in FIGS. 26-31, the threaded screw shank 502 may be tapered, with a greater diameter adjacent the arched head 900 and a lesser diameter adjacent a lower end portion of the threaded screw shank 502. In other embodiments, for example as illustrated in FIG. 32, the threaded screw shank 502 may be formed having a consistent diameter along the length of the shank 502. A consistent diameter may be easier to manufacture using standard lathe and milling operations, as will be understood by one of ordinary skill in the art.

In some embodiments, the threaded screw shank 502 may include one or more cutting flutes 501 positioned at a lower end portion of the shank 502. These one or more cutting flutes 501 may facilitate screw entry into the bone during implantation. Although FIG. 32 illustrates a cutting flute 501 positioned on a threaded screw shank 502 having a consistent diameter, in other embodiments threaded screw shanks 502 having tapered diameters, for example as illustrated in FIG. 26-31, may include one or more cutting flutes 501 positioned thereon.

The arched configuration seen in the arched head 900 can provide the ability to achieve a desired orientation, given the natural curvatures of the spine and the different sizes of vertebrae. When pedicle screws are placed in pedicles, they may be orientated at slightly different angles, such that the rods connecting these screws may be slightly bent or curved before placement.

As illustrated in the embodiments shown in FIGS. 26-32, the arcuate slide 901 can be positioned on an upper surface of the arched head 900 so as to engage with the bottom flange portions 307 of the rails 304 positioned on a lower surface of the tulip head 300. In addition to the arcuate slide 901, the arched head 900 of the pedicle screw may also include a pair of channels 903 positioned below the slide 901 on opposite sides of the arched head 900. These channels 903 may also have an arcuate shape, and may be shaped and positioned so as to receive and engage the bottom flange portions 307 of the rails 304 positioned on the lower surface of the tulip head 300. The arrangement of the channels 903 below the slide 901 and above the rail supports 904 may allow the bottom flange portions 307 of the rails 304 of the tulip head to securely engage the tulip head 300 with the arched head 900 of the pedicle screw. During implantation, the surgeon may need to manipulate the modular pedicle screw assembly in a relatively rough manner in order to achieve proper alignment and to secure the one or more rods. This manipulation may otherwise compromise the connection between the tulip head 300 and the arched head 900 of the pedicle screw. The inclusion of channels 903 and rail supports 904 strengthens this connection to allow for such manipulation without risking disconnection of the tulip head 300 from the arched head 900 of the pedicle screw. The channels 903 may additionally help to minimize the size of the arched screw head 900.

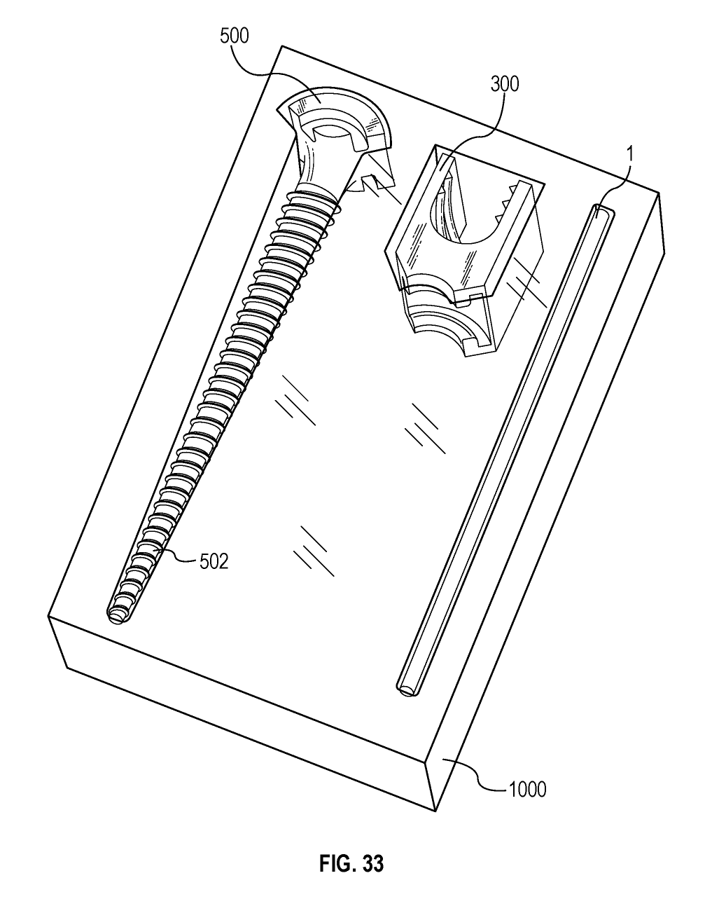

The present disclosure is also directed to a modular pedicle screw assembly kit, for example as illustrated in FIG. 33. In an embodiment, the kit may include a container 1000 housing each of the components of a modular pedicle screw assembly 20 needed to stabilize vertebrae of a spine, including a pedicle screw 500, a modular head 300, and a spinal rod 1. Although illustrated as a simplified, open-faced, molded rectangular container in FIG. 33, in various embodiments the container 1000 may take any shape, form, or size as is useful in the storage and presentation of a modular pedicle screw assembly, as will be readily understood by one of ordinary skill in the art.

The modular head 300 may be any tulip head or modular head as described herein, for example as illustrated in FIGS. 2, 4A-4D, 6A-6B, 9A-9E, 15-20, and 26-31, and as described in detail above. The pedicle screw 500 may include any base 200 or screw head 600, 900 as illustrated and described herein. In some embodiments, the container 1000 may include one or more of each of the pedicle screw 500, modular head 300, and spinal rod 1, in any number or combination as needed for a particular vertebral stabilization procedure. The container 1000 may be formed in any shape or size, and in any material, appropriate and effective for the sterile storage of the modular pedicle screw assembly components, as will be readily understood by one of ordinary skill in the art.

The present application is a Continuation in Part of and claims priority to International Application No. PCT/US2018/025300, filed Mar. 29, 2018, titled “MODULAR PEDICLE SCREW ASSEMBLIES AND ASSOCIATED METHODS,” which claims priority to, and the benefit of, U.S. Provisional Patent Application No. 62/479,285, filed Mar. 30, 2017, titled “MODULAR PEDICLE SCREW ASSEMBLIES AND ASSOCIATED METHODS,” each of which is incorporated herein by reference in its entirety.

Although the technology herein has been described with reference to particular embodiments, it is to be understood that these embodiments are merely illustrative of the principles and applications of the present technology. It is therefore to be understood that numerous modifications may be made to the illustrative embodiments and that other arrangements may be devised without departing from the spirit and scope of the present technology as defined by the appended claims.

That claimed is:

1. A pedicle screw for use in supporting a modular head and a spinal rod connected to the modular head, the pedicle screw comprising:
a threaded shank;
a screw head positioned at an upper end portion of the threaded shank, the screw head having:
an arcuate upper surface forming a convex slide positioned to engage a pair of rails positioned on a lower surface of the modular head, and
a pair of arcuate channels, each of the pair of arcuate channels positioned below the arcuate upper surface on an opposite side of the screw head, each of the pair of arcuate channels positioned to receive a bottom flange portion of the pair of rails of the modular head therein so as to secure the modular head on the pedicle screw; and
a screw neck connecting the threaded shank and the screw head, the screw neck positioned to provide a tapered transition between the screw head and the threaded shank.

2. The pedicle screw of claim 1, wherein the screw neck has a circular cross-section.

3. The pedicle screw of claim 2, wherein the screw neck is shaped conically so as to provide the tapered transition between the screw head and the threaded shank.

4. The pedicle screw of claim 1, wherein the threaded shank, the screw head, and the screw neck are integrally formed.

5. The pedicle screw of claim 1, wherein the threaded shank is formed with one of a longitudinally tapered shank or a shank having a consistent diameter.

6. The pedicle screw of claim 5, wherein the threaded shank comprises one or more cutting flute positioned at a lower end portion thereof, the cutting flute positioned so as to facilitate entry of the threaded shank into a bone.

7. A pedicle screw assembly for use in supporting a spinal rod, the assembly comprising:
a pedicle screw having:
a threaded shank;
a screw head positioned at an upper end portion of the threaded shank, the screw head having:
an arcuate upper surface forming a convex slide positioned to engage a pair of rails positioned on a lower surface of a modular head, and
a pair of arcuate channels, each of the pair of arcuate channels positioned below the arcuate upper surface on an opposite side of the screw head, each of the pair of arcuate channels positioned to receive a bottom flange portion of the pair of rails of the modular head therein so as to secure the modular head on the pedicle screw, and
a screw neck connecting the threaded shank and the screw head, the screw neck positioned to provide a tapered transition between the screw head and the threaded shank; and
the modular head having a rod recess positioned on an upper surface of the modular head so as to receive the spinal rod, and the lower surface of the modular head being positioned to define the bottom flange portion for engagement with the screw head,
the relative configuration of the screw head and the modular head arranged so that once engaged, the modular head is freely slidable along the arcuate upper surface of the screw head, but the screw head and modular head remain coupled to one another.

8. The pedicle screw assembly of claim 7, wherein the screw neck has a circular cross-section.

9. The pedicle screw assembly of claim 8, wherein the screw neck is shaped conically so as to provide the tapered transition between the screw head and the threaded shank.

10. The pedicle screw assembly of claim 7, wherein the threaded shank, the screw head, and the screw neck are integrally formed.

11. The pedicle screw assembly of claim 7, wherein the threaded shank is formed with one of a longitudinally tapered shank or a shank having a consistent diameter.

12. The pedicle screw assembly of claim 11, wherein the threaded shank comprises one or more cutting flute positioned at a lower end portion thereof, the cutting flute positioned so as to facilitate entry of the threaded shank into a bone.

13. A modular pedicle screw assembly kit for stabilizing vertebrae of a spine, the kit comprising:
a container;
a pedicle screw positioned in the container, the pedicle screw having:
a threaded shank;
a screw head positioned at an upper end portion of the threaded shank, the screw head having:
an arcuate upper surface forming a convex slide positioned to engage a pair of rails positioned on a lower surface of a modular head, and
a pair of arcuate channels, each of the pair of arcuate channels positioned below the arcuate upper surface on an opposite side of the screw head, each of the pair of arcuate channels positioned to receive a bottom flange portion of the pair of rails of the modular head therein so as to secure the modular head on the pedicle screw, and
a screw neck connecting the threaded shank and the screw head, the screw neck positioned to provide a tapered transition between the screw head and the threaded shank;
the modular head positioned in the container, the modular head having a rod recess positioned on an upper surface of the modular head so as to receive the spinal rod, and the lower surface of the modular head being positioned to define the bottom flange portion for engagement with the screw head; and
the spinal rod positioned in the container.

14. The kit of claim 13, wherein the screw head and the modular head are configured such that, once engaged, the modular head is freely slidable along the arcuate upper surface of the screw head, but the screw head and modular head remain coupled to one another.

15. The kit of claim 13, wherein the screw neck has a circular cross-section.

16. The kit of claim 15, wherein the screw neck is shaped conically so as to provide the tapered transition between the screw head and the threaded shank.

17. The kit of claim 13, further comprising a plurality of pedicle screws and a plurality of modular heads positioned in the container, the plurality of pedicle screws and the plurality of modular heads configured so as to engage the spinal rod.
